(19) United States
(12) Patent Application Publication
(10) Pub. No.: US 2004/0186063 A1
(43) Pub. Date: Sep. 23, 2004

(54) Conjugates of biologically active compounds, methods for their preparation and use, formulation and pharmaceutical applications thereof

(76) Inventors: Hans-Jurgen Gutke, Stuttgart (DE); Christian Flohr, Herrenberg (DE); Albert Beck, Karlsfeld (DE); Simona Margutti, Tubingen (DE); Mary Eggers, Ammerbuch (DE); Jan-Hinrich Guse, Tubingen (DE); Moussa Khobzaoui, Kusterdingen (DE); Michael Burnet, Kusterdingen (DE)

Correspondence Address: EDWARDS & ANGELL, LLP, P.O. BOX 55874, BOSTON, MA, 02205, US

(21) Appl. No.: 10/644,600

(22) Filed: Aug. 20, 2003

Related U.S. Application Data
(63) Continuation-in-part of application No. 10/367,105, filed on Feb. 14, 2003.
(60) Provisional application No. 60/357,434, filed on Feb. 15, 2002.

(51) Int. Cl. C07H 17/08; A61K 31/7048

(52) U.S. Cl. 514/28; 536/7.4

(57) ABSTRACT

This invention features a compound of the following formula:

T-(-L-C)ₘ,

T is a transportophore, L is a bond or a linker having a molecular weight up to 240 dalton, C is a non-antibiotic therapeutic agent, and m is 1, 2, 3, 4, 5, 6, 7, or 8, in which the transportophore has an immune selectivity ratio of at least 2, the transportophore is covalently bonded to the non-antibiotic therapeutic agent via the bond or the linker, and the compound has an immune selectivity ratio of at least 2.

### SUMMARY

Successful therapy with a pharmaceutical agent requires that the agent satisfy numerous requirements imposed by the physiology of the host and of the disease or condition. The requirements include: (i) adequate ability to interact with the target receptor(s); (ii) appropriate physical properties for presence at the location of the receptors in concentrations that permit the interactions noted above; (iii) appropriate physical properties to allow the agent to enter the body and distribute to the location of the receptors by any means; (iv) sufficient stability in fluids of the body; (v) the absence of toxic effects in compartments where the therapeutic agent is most concentrated, or in any other compartment where the therapeutic agent is located; and (vi) the absence of sequestration into non-physiological compartments and so on.

In general, these compounding requirements limit the nature of pharmaceutical compounds that have utility in vivo and thus reduce the probability of discovering adequately active molecules from de novo starting points. In response to these constraints, significant effort has been applied to the question of predicting ideal physical properties for pharmaceutical molecules. Authors such as Lipinski (Lipinski et al., 2001) have described rules of therapeutic agent design which, amongst other parameters, predicts that ideal therapeutic agents will have few functions such as hydroxy groups, a molecular weight below 500 Da, mild basicity, and moderate lipophilicity (log P<5) (Lipinski et al., 2001). Unfortunately, these parameters are too general to inform the direct synthesis of highly bioavailable compounds. Furthermore, these requirements are not helpful for larger molecule chemistry (MW>500) such as the compounds disclosed here.

Recently, improvements in the technology of synthetic chemistry and molecular biology have allowed the testing of large numbers of molecules and the discovery of many ligands with adequate affinity to their targets to have some potential in vivo. Many such molecules prove inadequate on in vivo testing largely due to the manifold, stringent, and often conflicting (i.e. stability without toxicity) requirements outlined above.

In addition to the difficulties facing many new molecules, many existing molecules in clinical use also exhibit inadequate properties of uptake, distribution, stability and toxicity (Lipinski et al. 2001). These observations demonstrate, that in general, deficiencies in uptake, distribution, and stability result in inadequate therapy from existing molecules and inadequate and uneconomical probabilities of success in the discovery of new molecules.

Such problems often fall within the scope of therapeutic agent delivery—a discipline which combines many aspects of formulation with techniques for introducing the agent into the host body. Delivery methods are frequently designed to permit passage through a single barrier (i.e. the skin) (WO 01/13957) or the intestine (WO 01/20331) after which the agent must again conform with the general requirements above in order to act at the in vivo target. Certain delivery strategies involve a physical preparation such as liposomes (Debs et al. 1990; Jaafari, Foldvari, 2002) or anti-body conjugates (Everts et al., 2002) which further direct the molecules within the host body. Others rely on the addition of cationic lipids to formulations, the use of transport proteins as a route of uptake (WO 01/20331). The use of transport processes deliberately in therapeutic agent design is perhaps best illustrated by the nucleoside therapeutic agents, which to varying degrees, are taken up as metabolites and whose transport to mitochondria is a major cause of toxicity (WO 98/29437) For example, see European Patent No. 0009944B 1, European Patent No. 0044090A3, and Japanese Patent No. 05163293. Such methods may enhance performance in therapy or reduce toxicity but they increase cost and require direct introduction into the blood stream which is impractical in chronic use.

More preferable would be small molecules that possess the appropriate structures and properties to mediate efficient uptake and stability. Such small molecules would ideally be able to carry a range of therapeutic agents of varying properties such that they could be commercialized in more than one indication. However, there is a requirement that they be inactive and stable enough to ensure that the cargo molecule is carried in the periphery (Harada et al. 2000).

The present invention represents a significant advance in that it provides for a means of improving the bioavailability and efficacy of a variety of molecules in vivo using a series of rational and facile assays to select desirable compounds based on known pharmacophores or pharmaceutical lead structures that have not been optimized for in vivo action.

### SUMMARY

The invention relates to a compound useful for enhancing efficacy of a therapeutic agent, a method for identifying such a compound, and a method of treating diseases including inflammation, graft rejection, infection, cancer, allergies, metabolic cardiovascular, pulmonary, dermatological, rheumatological and hepatic diseases. The invention further comprises compositions and formulations selected using the method and applications for same.

The invention provides for a method for identifying compounds that act as carriers or “transportophores” (i.e., a transport mediating molecule) that when combined, either directly or via a linker, to a wide variety of therapeutic agents, improves one or more of the following characteristics of the agent: ease of formulation, gastric stability, bioavailability, stability, disposition, elimination, half life, efficacy, safety, duration of action and selectivity.

In one aspect, this invention features a compound of the following formula (or referred to as T-L-C hereinafter):

T(L-C)ₘ,

wherein T is a transportophore, L is a bond or a linker having a molecular weight up to 240 dalton, C is a non-antibiotic therapeutic agent, and m is 1, 2, 3, 4, 5, 6, 7, or 8, in which the transportophore has an immune selectivity ratio of at least 2, the transportophore is covalently bonded to the non-antibiotic therapeutic agent via the bond or the linker, and the compound has an immune selectivity ratio of at least 2. Note that when there are more than one L or C moieties (i.e., m is greater than 1), the L moieties or the C moieties, independently, can be the same or different. The same rule applies to other similar situations.

The transportophore can be a metabolite, a natural product, a metabolite mimic, a metabolite derivative (e.g., a sugar, amino, or peptide derivative), a fatty acid, a bile acid, a vitamin, a nucleobase, an alcohol, or an organic acid or base, a portion of which resembles and is recognized as a substrate for transport protein(s). It can be an amphiphilic molecule having a pKa value of 6.5 to 9.5, or a cyclic or heterocyclic molecule (e.g., lactone, lactam, ether, cyclic acetal or hemi-acetal). The cyclic or heterocyclic molecule can have an attached sugar. The cyclic or heterocyclic molecule can be a macrolactone or macroether, including a macrolactone or macroether having an attached sugar. The cyclic or heterocyclic molecule can also be a macrolide or ketolide having an amino sugar, including a macrolide having mono-, di-, or tri-basic groups (e.g., an amine). In some embodiments, the macrolide has no intrinsic antibacterial activity (inactive at 50 uM or higher concentrations when tested against Bacillus in vitro see protocol) and a pKa value of less than 9.0 (e.g., 8.5, 8.0, 7.5, 7.0, or any number in between).

In some embodiments, the compound has the following formula (in which a bond, drawn without any attached groups, means a methyl group. The same rule applies to other similar situations):

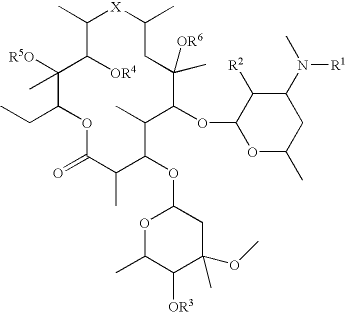

Wherein,

X═N(R⁷)—CH₂

CH₂—N(R⁷)

C(═O)

C(═NOR⁸)

CH(OR⁹)

CH(NR¹⁰R¹¹)

C(═NR¹²)

OC(═O)

C(═O)O

Y=independently,

Linker (as defined below)

z=C(═O)—

CH(R¹⁶)

R¹=H

CH₃

(C₂-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

Y—R¹³

C(═O)—Y—R¹⁵

C(═O)—R¹⁵

R²=H

(1′,2′-cis)-OH

(1′,2′-trans)-OH

(1′,2′-cis)-OR¹⁵

(1′,2′-trans)-OR¹⁵

(1′,2′-cis)-SH

(1′,2′-cis)-S—Y—R¹³

or the R¹ and R² bearing atoms are connected via a —OC(═O)CHR¹⁶— element

R³=H

C(═O)—Y—R¹⁵

C(═O)—R¹⁵

R⁴=H

C(═O)—Y—R¹⁵

C(═O)—R¹⁵

R⁵=H

or R⁴, R⁵ are connected by Z

R⁶=H

CH₃

R⁷=H

CH₃

Y—R¹³

C(═O)—Y—R¹⁵

C(═O)—R¹⁵

R⁸=H

Y—R¹³

R¹³

C(═O)—R¹⁷

(C₁-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

wherein alkyl, alkenyl, alkynyl, aryl, and heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, —NR¹⁸R¹⁹, R¹⁸C(═O)—, R¹⁸C(═O)O—, R¹⁸OC(═O)O—, R¹⁸NHC(═O)—, R¹⁸C(═O)NH—, R¹⁸R¹⁹NC(═O)— and R¹⁸OC(═O)—

R⁹=H

(C₁-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

wherein alkyl, alkenyl, alkynyl, aryl, and heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, —NR¹⁸R¹⁹, R¹⁸C(═O)—, R¹⁸C(═O)O—, R¹⁸OC(═O)O—, R¹⁸NHC(═O)—, R¹⁸C(═O)NH—, R¹⁸R¹⁹NC(═O)— and R¹⁸OC(═O)—

R¹⁰, R¹¹=independently H

(C₁-C₁₀)alkyl

(C₁-C₁₀)alkenyl

(C₁-C₁₀)akynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

or R¹⁰=H and R¹¹=—Y—R¹³

C(═O)—Y—R¹⁵, —C(═O)—R¹⁵

R¹²═H

(C₁-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

Y—R¹³

R¹³=R¹⁵=independently, therapeutic agent

R¹⁶=H

CH₃

(C₂-C ₁₀)alkyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

Y—R¹³

R¹⁷=O—R²⁰-aryl

optionally substituted by —X′-Y-therapeutic agent, X′-therapeutic agent wherein X′ is

S

O

NH

R¹⁸, R¹⁹ independently H

(C₁-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

R²⁰=independently

Halogen

(C₁-C₃)alkyl

NO₂

CN

OCH₃

N(CH₃)₂

N₃

SH

S(C₁-C₄)alkyl

In some other embodiments, the compound has the following formula:

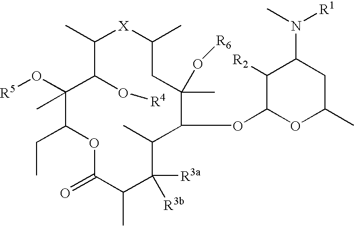

Wherein,

X=N(R⁷)—CH₂

CH₂—N(R⁷)

C(═O)

C(═NOR⁸)

CH(OR⁹)

CH(NR¹⁰R¹¹)

C(═NR¹²)

OC(═O)

C(═O)0

Y=independently, Linker (as defined below)

Z=C(═O)—

CH(R¹⁶)

R¹=H

CH₃

(C₂-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

Y—R¹³

C(═O)—Y—R¹⁵

C(═O)—R¹⁵

S(═O)ₖ(C₁-C₁₀)alkyl

S(═O)ₖ(C₁-C₁₀)alkenyl

S(═O)ₖ(C₁-C₁₀)alkynyl

S(═O)ₖ(C6-C₁₀)aryl

S(═O)ₖ(C₂-C₉)heteroaryl

S(═O)ₖ—Y—R¹⁵

S(═O)ₖ—R¹⁵

wherein k is 0, 1 or 2, and alkyl, alkenyl, alkynyl, cycloalkyl, heterocycloalkyl, aryl and heteroaryl can optionally be substituted by one to three halogen, cyano, hydroxy, (C₁-C₄)alkyloxy, nitro, (C₁-C₆)alkyl, (C₁-C₆)alkenyl, (C₁-C₆)alkynyl, (C₃-C₇)cycloalkyl, (C₁-C₆)heterocycloalkyl, (C₆-C₁₀)aryl, (C₁-C₉)heteroaryl, NR¹⁸R¹⁹, R¹⁸C(═O)—, R¹⁸C(═O)O—, R¹⁸OC(═O)—, R¹⁸C(═O)NH—, R¹⁸NHC(═O)—, R¹⁸R¹⁹NC(═O)— and R¹⁸OC(═O)—O—

R²=H

(1′,2′-cis)-OH

(1′,2′-trans)-OH

(1′,2′-cis)-OR¹⁵

(1′,2′-trans)-OR¹⁵

(1′,2′-cis)-SH

(1′,2′-cis)-S—Y—R¹³

or the R¹ and R² bearing atoms are connected via a —OC(═O)CHR¹⁶-element

R^{3a}, R^{3b}=independently H

R¹

OH

OR¹¹

NR¹⁰R¹¹

or R^{3a}=R^{3b}=(═O),

(═NR¹)

O(CH₂)ₖO— wherein k is 2 or 3

R⁴═H

C(═O)—Y—R¹⁵

C(═O)—R¹⁵

R⁵=H

or R⁴, R⁵ are connected by —Z—

R⁶=H

CH₃

R⁷=H

CH₃

Y—R¹³

C(═O)—Y—R¹⁵

C(═O)—R¹⁵

R⁸=H

Y—R¹³

C(═O)—R¹⁷

R⁹=H

(C₁-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

R¹⁰,R¹¹=independently H

(C₁-C₁₀)alkyl

(C₁-C₁₀)alkenyl

(C₁-C₁₀)akynyl

(C₃-C₁₀)cycloalkyl

(C₁-C₉)heterocycloalkyl

(C₆-C₁₀)aryl

(C₂-C₉)heteroaryl

wherein alkyl, alkenyl, alkynyl, cycloalkyl, heterocycloalkyl, aryl, heteroaryl are optionally substituted by one to three halogen, cyano, hydroxy, (C₁-C₄)alkyloxy, nitro, (C₁-C₆)alkyl, (C₁-C₆)alkenyl, (C₁-C₆)alkynyl, (C₃-C₇)cycloalkyl, (C₁-C₆)heterocycloalkyl, (C₆-C₁₀)aryl, (C₁-C₉)heteroaryl, NR¹⁸R¹⁹, R¹⁸C(═O)—, R¹⁸C(═O)O—, R¹⁸OC(═O)—, R¹⁸C(═O)NH—, R¹⁸NHC(═O)—, R¹⁸R¹⁹NC(═O)— and R¹⁸OC(═O)—O—

or R¹⁰=H and

R¹¹=Y—R¹³

C(═O)—Y—R¹⁵

C(═O)—R¹⁵

S(═O)ₖ(C₁-C₁₀)alkyl

S(═O)ₖ(C₁-C₁₀)alkenyl

S(═O)ₖ(C₁-C₁₀)alkynyl

S(═O)ₖ(C₆-C₁₀)aryl

S(═O)ₖ(C₂-C₉)heteroaryl

S(═O)ₖ—Y—R¹⁵

S(═O)ₖ—R¹⁵

wherein k is 0, 1 or 2 and alkyl, alkenyl, alkynyl, cycloalkyl, heterocycloalkyl, aryl and heteroaryl can be substituted as defined above.

R¹²=H

(C₁-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

Y—R¹³

R¹³═R¹⁵=independently therapeutic agent

R¹⁶=H

CH₃

(C₂-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

(C₁-C₄)alkyliden-NR¹⁸R¹⁹

Y—R¹³

R¹⁷═O—R²⁰-aryl

optionally substituted by —X′—Y-therapeutic agent, X′-therapeutic agent wherein X′ is

S, O, NH

R¹⁸, R¹⁹=independently H

(C₁-C₁₀)alkyl

(C₁-C₁₀)alkenyl

(C₁-C₁₀)alkynyl

(C₁-C₈)[(C₁-C₄)alkoxy]alkyl

(C₁-C₈)[(C₁-C₄)alkoxy]alkenyl

(C₆-C₁₀)aryl-(C₁-C₅)alkyl

(C₂-C₉)heteroaryl-(C₁-C₅)alkyl

R²⁰=independently,

Halogen

(C₁-C₃)alkyl

NO₂

CN

OCH₃

N(CH₃)₂

N₃

SH

S(C₁-C₄)alkyl

In still some other embodiments, the compound has the following formula:

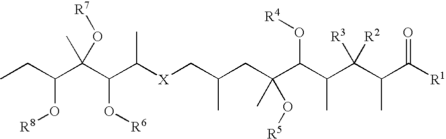

Wherein,

X=N(R⁹)—CH₂

CH₂—N(R⁹)

C(═O)

C(═NOR¹⁰)

C(OR¹¹)H

CH(NR¹²R¹³)

C(═NR¹⁴)

OC(═O)

C(═O)O

Y=independently, Linker (as defined below)

R¹=OR¹⁷

NR¹⁷R¹¹,

or R¹ is connected to the oxygen bearing R⁴ or R⁵ forming a lactone or is connected to a suitable substituent in R² forming a lactone or lactam.

R²=O-2-cladinosyl

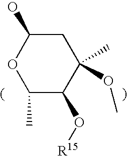

H

X′, wherein X′=halogen

azido

nitro

cyano

OR¹⁷

OR²²

NR¹⁷R¹⁸

SR¹⁷(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰C(═O)O—, —Y-therapeutic agent or -therapeutic agent

R³=H

(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, R²⁰R²¹N—

R²⁰=O-2-desosaminyl

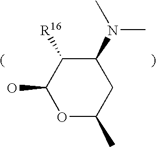

H

C(═O)R¹⁷

Y-therapeutic agent

therapeutic agent

S(═O)₂R¹⁷ providing R¹⁷ is not hydrogen

C(═O)NR¹⁷R¹⁸(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y-therapeutic agent or -therapeutic agent

or R⁴ is connected to a suitable R² containing a N or a O by —C(═O), S(═O), wherein n=1 or 2, —CR²⁰R¹⁷—, CR²⁰(—Y-therapeutic agent)-, —CR²⁰(-therapeutic agent)- forming in dependence of R² a 6 or 7-membered ring

R⁵=R²⁰

C(═O)R²⁰

or R⁴, R⁵ are connected by C(═O), S(═O)ₙ wherein n 1 or 2, —CR²⁰R¹⁷—, CR²⁰(—Y-therapeutic agent)-, —CR²⁰(-therapeutic agent)-

R⁶, R⁸=independently H

(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²¹R²¹NC(═O)—, and R²¹OC(═O)O—, —Y-therapeutic agent or -therapeutic agent,

or R⁶, R⁸=independently —C(═O)R¹⁷, —Y-therapeutic agent, -therapeutic agent, —S(═O)₂R¹⁷ providing R¹⁷ is not hydrogen, —C(═O)NR¹⁷R¹⁸

R⁷=H

(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y-therapeutic agent or -therapeutic agent

or two of each R⁶, R⁷, R⁸ are connected by —C(═O), S(═O)ₙ wherein n=1 or 2, —CR²⁰R¹⁷—, CR²⁰(—Y-therapeutic agent)-, —CR²⁰(-therapeutic agent)-

R⁹=H

CH₃

Y-therapeutic agent

therapeutic agent

(C₁-C₆)alkyl

(C₁-C₆)alkenyl

(C₁-C₆)alkynyl,

wherein alkyl, alkenyl, alkynyl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y-therapeutic agent or therapeutic agent

R¹⁰=C(═O)-aryl

therapeutic agent

H

(C₁-C₆)alkyl

(C₁-C₆)alkenyl

(C₁-C₆)alkynyl,

wherein alkyl, alkenyl, alkynyl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y-therapeutic agent or -therapeutic agent

R¹¹=H

(C₁-C₆)alkyl

(C₁-C₆)alkenyl

(C₁-C₆)alkynyl,

wherein alkyl, alkenyl, alkynyl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, R²⁰OC(═O)O—, —Y-therapeutic agent or -therapeutic agent,

or R¹¹=—Y-therapeutic agent, -therapeutic agent, —C(═O)R¹⁷

R¹², R¹³=independently H

(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl,

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²¹NC(═O)—, R²⁰C(═O)NH—, R²⁰R²¹NC(═O)—, R²⁰OC(═O)O—, —Y-therapeutic agent or -therapeutic agent,

or R¹², R¹³=independently —C(═O)R¹⁷, —Y-therapeutic agent, -therapeutic agent, —S(═O)₂R¹⁷ providing R¹⁷ is not hydrogen, —C(═O)NR¹⁷R¹⁸

R¹⁴=therapeutic agent

H

(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, R²⁰OC(═O)O—, —Y-therapeutic agent or -therapeutic agent

R¹⁵=H

C(═O)R¹⁷

Y-therapeutic agent

therapeutic agent

S(═O)₂R¹⁷ providing R¹⁷ is not hydrogen

C(═O)NR¹⁷R¹⁸

(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl,

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y-therapeutic agent or -therapeutic agent

R¹⁶=H

OR¹⁷

OR²²

R¹⁷, R¹⁸=independently H

(C₁-C₆)alkyl

(C₁-C₆)alkenyl

(C₁-C₆)alkynyl

(C₃-C₁₀)cycloalkyl

(C₁-C₉)heterocycloalkyl

(C₆-C₁₀)aryl

(C₁-C₉)heteroaryl

wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²¹NHC(═O)—, R²⁰C(═O)NH—, R²⁰R²¹NC(═O)—, and R²⁰OC(═O)O—, —Y-therapeutic agent or -therapeutic agent

or provided that connected to a nitrogen, R¹⁷, R¹⁸ may form a cyclic structure of 4 to 7 members (including the nitrogen). R¹⁷ and R¹⁸ then can represent a fragment from the type of —[C(AB)]ₘ—Ξₙ-[C(DE)]ₒ-Ψₚ-[C(GJ)]_{q} wherein m, n, o, p and q independently are 0, 1, 2, 3, 4, 5, or 6, Ξ and Ψ independently are —O—, —S—, —NK— and A, B, D, E, G, J, and K independently are hydrogen, (C₁-C₄) alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(=)-, R²⁰NHC(═O)—, R²⁰C(═O)NH—, R²⁰R²¹NC(═O)—, and R²⁰OC(═O)O—

R²⁰, R²¹=independently H

(C₁-C₆)alkyl

R²²=C(═O)R¹⁷

Y-therapeutic agent

therapeutic agent

S(═O)₂R¹⁷ providing R¹⁷ is not hydrogen, —C(═O)NR¹⁷R¹⁸.

In further embodiments, the compound has the following formula:

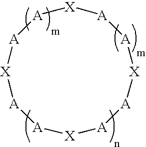

Wherein

m=independently, 0, 1, 2, 3

n=0-7

X=independently

O

S

Se

NR¹

PR¹

with the proviso, that at least one X=−NR¹—

A independently

CH₂

CHR²

CR²R³

C(═O)

with the proviso, that at least one X=—NR¹— is not an amide

R¹=independently,

H

(C₁-C₁₀)alkyl optionally substituted by fluoro, cyano, R⁴, R⁴O₂C, R⁴C(═O)NH and R⁴S(═O)ₖ wherein k is 0, 1 or 2

R⁴C(═O), R⁴S(═O)ₖ wherein k is 0, 1 or 2

R², R³=independently NH₂

NHR¹

NR¹R⁵

OH,

OR⁴

R⁴C(═O) (C₁-C₆)alkyl

(C₂-C₁₂)alkenyl

(C₂-C₁₂)alkynyl

(C₃-C₁₀)cycloalkyl(C₁-C₆)alkyl

(C₂-C₉)heterocycloalkyl(C₁-C₆)alkyl

(C₆-C₁₀)aryl(C₁-C₆)alkyl

(C₂-C₉)heteroaryl(C₁-C₆)alkyl,

wherein the alkyl, alkenyl, alkynyl, cycloalkyl, heterocycloalkyl, aryl, and heteroaryl groups are optionally substituted by one to three halo, (C₁-C₄)alkoxy, hydroxy, nitro, cyano, —C(═O)—OR⁸, —C(═O)N(H)R⁸, (C₆-C₁₀)aryl, (C₂-C₉)heteroaryl, N*R⁵R⁶R⁷ wherein * is no or a positive charge, one or two of R², R³ can be a directly coupled therapeutic agent

R⁴=independently

NH₂

NHR⁹

NR⁹R⁵

OH

OR⁹

(C₁-C₆)alkyl

(C₂-C₁₂)alkenyl

(C₂-C₁₂)alkynyl

(C₃-C₁₀)cycloalkyl(C₁-C₆)alkyl

(C₂-C₉)heterocycloalkyl(C₁-C₆)alkyl

(C₆-C₁₀)aryl(C₁-C₆)alkyl

(C₂-C₉)heteroaryl(C₁-C₆)alkyl,

wherein the alkyl, alkenyl, alkynyl, cycloalkyl, heterocycloalkyl, aryl, and heteroaryl groups are optionally substituted by one to three halo, (C₁-C₄)alkoxy, hydroxy, nitro, cyano, R⁸, —C(═O)—OR⁸, —C(═O)N(H)R⁸, (C₆-C₁₀)aryl, (C₂-C₉)heteroaryl, N*R⁵R⁶R⁷ wherein * is no or a positive charge, or therapeutic agent

R⁵, R⁶=independently H

(C₁-C₆), optionally substituted by hydroxy

(C₆-C₁₀)aryl

(C₂-C₉)heteroaryl

R⁷=independently

lone electron pair

CH₃

C₂H₅

C₃H₇

CH₂—C₆H₅

R⁸=independently,

therapeutic agent

R⁹=independently,

(C₁-C₆) alkyl

(C₂-C₁₂)alkenyl

(C₂-C₁₂)alkynyl

(C₃-C₁₀)cycloalkyl(C₁-C₆)alkyl

(C₂-C₉)heterocycloalkyl(C₁-C₆)alkyl

(C₆-C₁₀)aryl(C₁-C₆)alkyl or

(C₂-C₉)heteroaryl(C₁-C₆)alkyl,

wherein the alkyl, alkenyl, alkynyl, cycloalkyl, heterocycloalkyl, aryl, and heteroaryl groups are optionally substituted by one to three halo, (C₁-C₄)alkoxy, hydroxy, nitro, cyano, R⁸, —C(═O)—OR⁸, —C(═O)N(H)R⁸, (C₆-C₁₀)aryl, (C₂-C₉)heteroaryl, N*R⁵R⁶R⁷ wherein * is no or a positive charge, or therapeutic agent.

Preferred molecules can be compounds that are recognized by a transport enzyme in the membrane of the cell of the tissue that the drug is is to target. This can be a molecule that fulfills the structural requirements in order to be recognized by an oligo-peptide transporter.

Compounds recognized by transport enzymes can be identified by performing a transport assay with the compound in question in cells expressing the transport protein in question, and comparing the level of compound accumulation with those from parallel uptake assays performed using cells which do not express the target transport protein.

According to well known models these structures may be as exemplified in the following sketches:

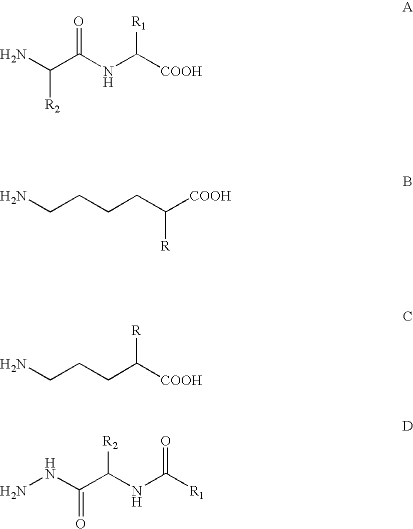

In these examples R (including R₁ and R₂) may represent a chemical residue that will modify the recognition by the transporting enzyme or at least not inhibit it. R may be comprised of the therapeutic agent that is to be delivered or the pharmaceutical entity is for example an amino acid itself as in example A.

Necessary for transport through an oligopeptide transporter seems to be a basic group spaced 4 or 5 bonds from an hydrogen bond accepting group like preferably carboxylate (example A-C) or less preferred amide (example D).

Example A: R₁ and R₂ are hydrogen or lower alkyl, branched or linear from C₁ to C₅, or benzyl or p-hydroxy benzyl, or hydroxy or mercapto methyl, or any group responsible for the desired pharmacological effect.

Example B: R can be the moiety responsible for the pharmacological effect, or the pharmacologically relevant group linked on the carbon chain by a chemical linker like an amide (amido-R═NH(C═O)—R′ (R′=pharmacologically relevant group)).

Example C: R can be the moiety responsible for the pharmacological effect, or the pharmacologically relevant group linked on the carbon chain by a chemical linker like an amide (amido-R═NH(C═O)—R′ (R′=pharmacologically relevant group)).

Example D: R2 can be hydrogen or lower alkyl, branched or linear from C1 to C5, or benzyl or p-hydroxy benzyl, or hydroxy or mercapto methyl, while R1 consists of the pharmacologically relevant therapeutic agent. Preferably the therapeutic agent would contain a carboxylic acid that by linking to the amino function of an amino acid hydrazide would obtain the general structure of example D.

Therapeutic agents and transportophores can be directly connected or via a linking element. This element typically is a bifunctional molecule of low molecular mass, which can react subsequently with the therapeutic agent and the transportophore. Ideally the therapeutic agent can be released from this linker under physiological conditions. This may be achieved oxidatively (i.e. by action of a cytochrome C), reductively (i.e. by action of NADH), hydrolytically (i.e. by action of a protease), or initiated by radicals (i.e. by the action of superoxide radicals). The mechanisms of therapeutic agent release are not limited to the above examples.

Linkers have the following formula:

F¹-M-F²

Where can be:

F¹, F²=independently a functional group, suitable to react with a counterpart in the therapeutic agent or in the transportophore. F¹ and F² are, but are not limited to

X¹ wherein X¹ is a halogen atom or a sulfonate ester or an other suitable leaving group;

C(═O)X² wherein X² is Cl, Br or I,

CHO;

C(═O)OR^{a} wherein R^{a} is (C₁-C₄)alkyl or aryl, optionally substituted by 1-5 halogen atoms;

C(═O)OC(═O)OR″ wherein R″ is (C₁-C₅)alkyl or (C₁-C₅)alkenyl;

OH;

NHR^{b} wherein R^{b} is H, (C₁-C₄)alkyl;

NCX³ wherein X³ is S or O;

C(═O)CR═CHR′, wherein R and R′ are independently —H, —CH₃, —Cl, —Br, —F, —O(C₁-C₄)alkyl, —C(═O)O(C₁-C₄)alkyl, —NO₂, —S(═O)ₖ(O)I(C₁-C₄)alkyl wherein k is 0, 1 or 2 and I is 0 or 1, SiR¹R²R³ wherein R¹, R² and R³ independently are (C₁-C₄)alkyl;

SX⁴ wherein X⁴ is —H, —Cl, —Sₖ(C₁-C₄)alkyl, Sₖ(C₆-C₁₀)aryl wherein k is 1 or 2.

F¹ and F² can be connected to form a cyclic anhydride or di- or trisulfide.

M is a spacing element which is, but is not limited to

(C₁-C₈)alkyl,

(C₁-C₈)alkenyl,

(C₁-C₈)alkynyl,

(C₃-C₁₀)cycloalkyl,

(C₆-C₁₀)aryl,

(C₂-C₉)heteroalkyl,

(C₂-C₉)heteroaryl.

Alkyl-, alkenyl, alkynyl, cycloalkyl, aryl or heteroaryl spacing elements are optionally substituted by (C₁-C₆)alkyl, 1-4 halogens, (C₁-C₄)alkoxy, (C₁-C₄)alkoxycarbonyl, hydroxy, amino, (C₁-C₄)alkylamino, (C₁-C₄)dialkylamino, (C₃-C₁₀)cycloalkyl, (C₁-C₆)alkylcarbonyloxy, (C₁-C₆)alkylcarbonylamido, (C₁-C₄)alkylamidocarbonyl, (C₁-C₄)dialkylamidocarbonyl, nitro, cyano, (C₁-C₄)alkylimino, mercapto and (C₁-C₄)alkylmercapto functions.

TABLE 1
Non-limiting examples of Linkers useful in the synthesis of T-L-C
molecules.*

| Donor linking | Recipient linking function | | |
|---|---|---|---|
| function | COOH | NH2 | OH |
| COOH | Ethylendiamine, | N-Methoxycarbonyl-4- | N-Methoxycarbonyl- |
| | Glycol, | hydroxyproline, | 4-hydroxyproline, |
| | (2-Aminoethyl)-(2- | Glycolic acid, β-Alanin, | Glycolic acid, |
| | hydroxyethyl)amino | β-hydroxy propanoic | β-Alanin, β-hydroxy |
| | | acid | propanoic acid |
| NH2 | N-Methoxycarbonyl-4- | Ethylendiamine, | 2,2-Dimethylsuccinic |
| | hydroxyproline, | 2,2-Dimethylsuccinic | acid, Succinic acid, |
| | Glycolic acid, | acid, | Glutaric acid, |
| | β-Alanin, β-hydroxy | Succinic acid, Glutaric | 2,4-Dimethylglutaric |
| | propanoic acid | acid, 2,4- | acid, |
| | | Dimethylglutaric acid, | Methyl dicarboxy- |
| | | Methyl | methylamin, |
| | | dicarboxymethylamino | 2-Aminoethyl-2- |
| | | | hydroxyethylamino |
| OH | N-Methoxycarbonyl-4- | 2,2-Dimethylsuccinic | β-Hydroxy propanoic |
| | hydroxyproline, | acid, Succinic acid, | acid, |
| | Glycolic acid, | Glutaric acid, | 2,2-Dimethylsuccinic |
| | β-Alanin, | 2,4-Dimethylglutaric | acid, Succinic acid, |
| | β-hydroxy propanoic | acid, | Glutaric acid, |
| | acid | Methyl dicarboxy- | 2,4-Dimethylglutaric |
| | | methylamin, | acid, |
| | | 2-Aminoethyl-2- | Methyl |
| | | hydroxyethylamino | dicarboxymethylamino |

*The donor linking function in vertical refers to a functional group on T; the recipient linking function in horizonal refers to a functional group on L; and the chemical groups in the boxes are the linkers (L).

The non-antibiotic therapeutic agent can be an anti-inflammatory agent (e.g. a p38 kinase inhibitor), an anti-viral agent, an anti-cancer agent, an immune-suppressant agent, a sterol synthesis modifying agent, agents active on protozoa, or an agent for treating a metabolic disease.

As used herein, an “immune selectivity ratio” is the ratio of the concentration of a compound in immune cells (e.g., neutrophils, monocytes, and lymphocytes) to the concentration of the compound in erythrocytic cells after the compound has been incubated in a mixture of blood cells including erythrocytes. A protocol of determining the immune selectivity ratio is described in Example 1.

A “therapeutic agent,” as used herein, is a molecule with pharmacological activity (e.g., a therapeutic agent, medicine, medicament, or active agent), a disease modification agent, or any other molecule that can be covalently attached to a transportophore via a bond or a linker which may have a desirable mode of action in immune or target cells. A therapeutic agent may be released from a compound described above in response to the enzyme activity or the physicochemical environment of the targeted cells. Thus, the therapeutic agent is selectively accumulated in a cell due to specific characteristics of the cell membranes, specific expression of membrane proteins, specific conditions within the cell, notably to expression of specific proteins such as granule proteins, binding sites in the cytoplasm, or other membrane bound or soluble proteins, and is thus trapped in the cell and therefore exhibits an enhanced or desired activity therein.

An “amphiphilic molecule,” as used herein, is a molecule having a hydrophilic (polar) and hydrophobic (non-polar) functional groups (e.g., atoms) or a combination of groups (or atoms). The pKa of this molecule is in the range of 6.5 to 9.5.

The term “cyclic” refers to a hydrocarbon cyclic ring including fully saturated, partially saturated, and unsaturated mono-, bi, and tri-cyclic rings having 4 to 34 ring atoms, preferably, 7 to 10, or 10 to 15 ring atoms. The term “heterocyclic” refers to a hydrocarbon cyclic ring including fully saturated, partially saturated, and unsaturated mono-, bi, and tri-cyclic rings having 4 to 34 ring atoms, preferably, 7 to 10, or 10 to 15 ring atoms having one or more heteroatoms, such as S, O, or N in each ring.

The term “sugar” refers to a mono-, di-, or tri-saccharide including deoxy-, thio-, and amino-saccharides. Examples of sugar include, but are not limited to, furanose and pyranose.

The terms “halogen” and “halo” refer to radicals of fluorine, chlorine, bromine or iodine.

The term “macrolactone” refers to a large lactone ring (i.e., cyclic ester) having at least 10 (if that is OK. Otherwise having 10 to 25)ring atoms.

The term “macrocyclic ether” refers to an ether having at least 10 (same as before) atoms.

The term “macrolide” refers to a chemical compound characterized by a large lactone ring (having at least 10 ring atoms) containing one or more keto and hydroxyl groups, or to any of a large group of antibacterial antibiotics containing a large lactone ring linked glycosidically to one or more sugars; they are produced by certain species of Streptomyces and inhibit protein synthesis by binding to the 50S subunits of 70S ribosomes. Examples include erythromycin, azithromycin, and clarithromycin.

The term “ketolide” refers to a chemical compound characterized by a large lactone ring (having at least 10 ring atoms) containing one or more keto groups.

The term “alkyl” (or “alkenyl” or “alkynyl”) refers to a hydrocarbon chain that may be a straight chain or branched chain, containing the indicated number of carbon atoms. For example, C₁-C₁₀ indicates that the group may have from 1 to 10 (inclusive) carbon atoms in it. Alkenyl groups and alkynyl groups have one or more double or triple carbon-carbon bonds, respectively, in the chain.

The term “aryl” refers to a hydrocarbon ring system (mono-cyclic or bi-cyclic) having the indicated number of carbon atoms and at least one aromatic ring. Examples of aryl moieties include, but are not limited to, phenyl, naphthyl, and pyrenyl.

The term “heteroaryl” refers to a ring system (mono-cyclic or bi-cyclic) having the indicated number of ring atoms including carbon atoms and at least one aromatic ring. The ring system includes at least one heteroatom such as O, N, or S (e.g., between 1 and 4 heteroatoms, inclusive, per ring) as part of the ring system. Examples of heteroaryl moieties include, but are not limited to, pyridyl, furyl or furanyl, imidazolyl, benzimidazolyl, pyrimidinyl, thiophenyl or thienyl, quinolinyl, indolyl, and thiazolyl.

The term “alkoxy” refers to an —O-alkyl radical.

The term “cycloalkyl” refers to a nonaromatic hydrocarbon ring system (mono-cyclic or bi-cyclic), containing the indicated number of carbon atoms.

The term “heterocycloalkyl” refers to a nonaromatic ring system (mono-cyclic or bi-cyclic), containing the indicated number of ring atoms including carbon atoms and at least one heteroatom such as O, N, or S (e.g., between 1 and 4 heteroatoms, inclusive, per ring) as part of the ring system.

“Alkyliden” is a bivalent alkyl group.

“Aryliden” is a bivalent aryl group.

“Erythrocytic cell” is a mature red blood cell that normally does not have a nucleus: it is a very small, circular disk with both faces concave, and contains hemoglobin, which carries oxygen to the body tissues.

The compounds described above include the compounds themselves, as well as their salts, if applicable. Such salts, for example, can be formed between a positively charged substituent (e.g., amino) on a compound and an anion. Suitable anions include, but are not limited to, chloride, bromide, iodide, sulfate, nitrate, phosphate, citrate, methanesulfonate, trifluoroacetate, and acetate. Likewise, a negatively charged substituent (e.g., carboxylate) on a compound can form a salt with a cation. Suitable cations include, but are not limited to, sodium ion, potassium ion, magnesium ion, calcium ion, and an ammonium cation such as tetramethylammonium ion.

In addition, some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z- double isomeric forms.

Further, the aforementioned compounds also include their N-oxides. The term “N-oxides” refers to one or more nitrogen atoms, when present in a compound, are in N-oxide form, i.e., N→O.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., treating a disease).

In another aspect, this invention features a method for treating an inflammatory disorder. The method includes administering to a subject in need thereof an effective amount of a compound described above, wherein the compound contains a non-antibiotic therapeutic agent that is an anti-inflammatory agent. Optionally, the method includes co-usage with other anti-inflammatory agents or therapeutic agents. The method is able to improve therapy by concentrating a compound preferentially in immune cells including neutrophils, monocytes, eosinophils, macrophage, alveolar macrophage, B and T-lymphocytes, NK cells, giant cells, Kupfer cells, glial cells, and similar target cells using a variety of means of concentrative compound uptake common to such cells. As such, the invention is advantageous in that selective concentration of compounds conforming to the definition of “therapeutic agent” above, can improve therapy and that, for the purposes of illustration only, concentration of agents in immune cells can confer improved characteristics on compounds with suitable modes of action for the treatment of inflammatory diseases.

In another aspect, the invention features a means of improving the action of a compound in vivo by reducing its exposure to the action of detoxification enzymes. Such reduced exposure is a result of the structure of the conjugate molecule causing it to be differently retained in the cells and organs of the organism and thus reducing or limiting the amount of material in a given metabolic compartment.

In another aspect, the invention provides for means to improve the action of a compound through improved retention in the cells and tissues of the organism such that it is less efficiently eliminated by the normal processes of circulation and filtration. Such avoidance of elimination is, at least in part, a consequence of efficient uptake into cells resulting in reduced concentrations of the drug being available from plasma.

In another aspect, the invention provides for a means of improving the action of a drug by assisting its uptake from the intestine through the overall effects on membrane permeability of the compound that are associated with the invention. Uptake from oral administration is a means of providing sustained exposure to the compound from the parts of the intestine to which it is permeable. Oral availability is not a property of all compounds.

This invention also features a method of treating a disease (e.g., an infectious disease including viral, fungal, or parasitic diseases, cancer, allergy, metabolic, cardiovascular, pulmonary, dermatological, rheumatological or immune disease). The method comprises administering to a subject in need thereof an effective amount of a compound described above, wherein the compound contains a non-antibiotic therapeutic agent (e.g., an anti-inflammatory agent (e.g. a p38 kinase inhibitor), an anti-viral agent, an anti-cancer agent, an immune-suppressant agent, a sterol synthesis modifying agent, agents active on protozoa, or an agent for treating a metabolic disease). Optionally, the method includes co-usage with other therapeutic agents. As described above, the method provides for means to improve therapy by concentrating a compound preferentially in any of the myeloid, hepatic, respiratory, epithelial, endothelial, other target and immune cells. Therefore, the invention is advantageous in that selective concentration of compounds conforming to the definition of “therapeutic agent” above, via the methods described, can improve therapy and that, for the purposes of illustration only, concentration of agents in immune cells can confer improved characteristics on compounds with suitable modes of action for the treatment of diseases of infectious, allergic, autoimmune, transplant, traumatic or neoplastic origin or association.

The present invention also features a pharmaceutical composition including at least one compound of this invention and a pharmaceutically acceptable carrier. Optionally, the pharmaceutical composition includes one or more other therapeutic agents.

This invention further features a method for making any of the compounds described above. The method includes taking any intermediate compound delineated herein, reacting it with any one or more reagents to form a compound of this invention including any processes specifically delineated herein.

In another aspect, this invention features a method of identifying a compound useful for enhancing efficacy of a therapeutic agent. The method includes incubating a compound in blood cells; separating immune cells from erythrocytic cells (e.g., by density gradient centrifugation, antibody mediated capture, lectin based capture, absorption to plastic, setting, simple centrifugation, peptide capture, activation mediated capture, or flow cytometry); and determining the ratio of the concentration of the compound in the immune cells to the concentration of the compound in the erythrocytic cells (e.g., by mass spectrometry, NMR, PET, fluorescence detection, infrared fluorescence, colorimetry, normal detection methods associated with gas chromatography, Fourrier transform spectrometry method, or radioactive detection); wherein the compound comprises a transportophore and a therapeutic agent, in which the transportophore is covalently bonded to the therapeutic agent via a bond or a linker. The therapeutic agent can be, for example, an anti-inflammatory agent (e.g. a p38 kinase inhibitor), an anti-viral agent, an anti-cancer agent, an immune-suppressant agent, a sterol synthesis modifying agent, agents active on protozoa, or an agent for treating a metabolic disease.

In still further another aspect, this invention features a method for delivering a therapeutic agent with a selective concentration. The method includes identifying a compound using the just-described method, and delivering the compound to a cell (e.g., a cell of the immune system).

Also within the scope of this invention are a composition having one or more of the compounds of this invention (optionally including one or more other therapeutic agents) for use in treating various diseases described above, and the use of such a composition for the manufacture of a medicament for the just-described use.

The invention provides several advantages. For example, a compound of this invention achieves one or more of the following improvements relative to a therapeutic agent itself: (i) improved uptake across the intestinal, jejunal, duodenal, colonic, or other mucosa; (ii) reduced first pass effect by mucosal oxygenases; (iii) reduced or altered detoxification by degradative enzymes of the body; (iv) reduced efflux; (v) selective accumulation of the therapeutic agent in one or more immune, fibroblast, hepatic, renal, glial, or other target cells; (vi) potential for hydrolytic or other forms of separation on a timescale compatible with therapy and the other desired disposition events; (vi) enhanced pharmacological effect in the target cells through greater concentration, sustained release, reduced substrate competition effect or other mechanisms; (vii) reduced or modified dose; (viii) modified route of administration; (ix) reduced or altered side effects; (x) alternative uses; and (xi) alternative formulations.

Other advantages, objects, and features of the invention will be apparent from the description and drawings, and from the claims.

### DESCRIPTION OF DRAWINGS

FIG. 1. Diagram of the essential entry process and subsequent cleavage of the prodrug. The conjugates of the drug are not only orally available, but also subsequently accumulated in immune cells where they act to achieve greater effect.

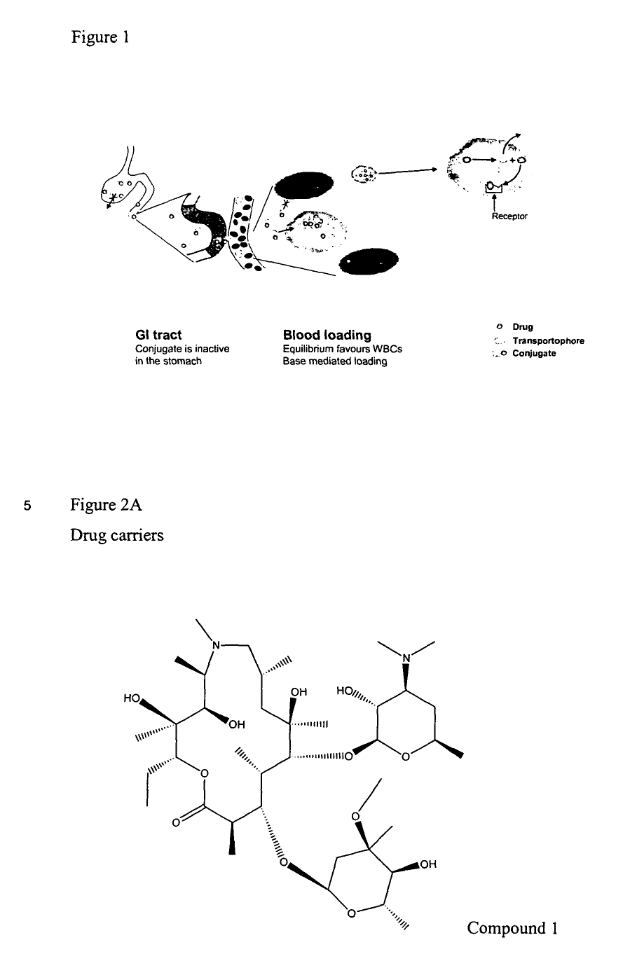

FIGS. 2A-2C. Structures of example macrocyclic drug carriers.

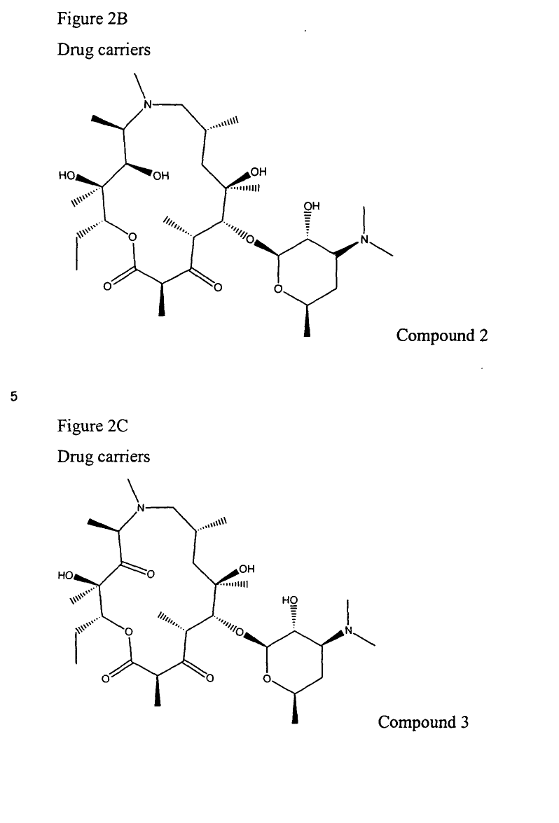

### DETAILED DESCRIPTION

The invention comprises compounds that are prodrugs with preferential uptake and activity in specific cells including immune cells. The invention thus provides for the rational improvement of therapeutic agents intended for action in inflammatory disease, infection, cancer, allergy, transplantation, cardiovascular, pulmonary, dermatological, rheumatological and metabolic disease. The invention also provides for methods to engender unoptimized molecules or those with activity only in vitro with improved properties in vivo through simple conjugation with molecules that meet the criteria outlined herein.

The compounds described herein can be prepared by methods known in the art, as well as by the synthetic routes disclosed herein. For example, one can react a transportophore having a reactive moiety with a therapeutic agent having another reactive moiety. One of the two reactive moieties is a leaving group (e.g., —Cl, OR) and the other is a derivatizable group (e.g., —OH, or —NH—). Then, the transportophore is covalently bonded to the therapeutic agent via a reaction between the two reactive moieties. In the case when a linker is present, each of the two reactive moieties, independently, is a leaving group or a derivatizable group, and each reacts with its reactive counterpart in the linker to form a covalent bond. Detailed routes including various intermediates are illustrated in the examples herein.

The chemicals used in the afore-mentioned methods may include, for example, solvents, reagents, catalysts, protecting group and deprotecting group reagents and the like. The methods described above may also additionally comprise steps, either before or after the steps described specifically herein, to add or remove suitable protecting groups in order to ultimately allow synthesis of the compound of the formulae described herein.

As can be appreciated by the skilled artisan, the synthetic routes herein are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

A therapeutic agent includes any with modes of action that include anti-inflammatory, anti-viral, immune suppressant, cytostatic, anti-parasitic, a sterol synthesis modifying, or metabolaregulatory action. The following is a non-exclusive list of potentially useful therapeutic agents in this invention.

Anti-inflammatory Therapeutic Agents

Kinase Inhibitors

PD 98059, AG 126, KN-93, RO 31-7549, RO 31-7549, RWJ 67657, Diacerein (KW-4800), VK-19911, VX-745, SB 203580, BIRB 796 BS, CNI-1493, EF5, KB-R7785, PD 169316, SB 202190, SCIO 469, Y-39041, E01428, SD-282, Thalidomide, RPR203494, RPR200765A, RPR132331, LY 294002, SP600125, GF 109203, Genistein, RO-31-8220, U 0126, Radicicol, SB 242235, GO696.

Statin Class Compounds

Simvastatin (cited here as an anti-inflammatory compound)

Reductase Inhibitors

HMGCoA reductase inhibitors. (See also sterol synthesis modifying agents)

Protease Inhibitors

For example: saquinavir, ritonavir, indinavir, nelfinavir, amprenavir, and lopinavir or those based on alternative non-peptidic scaffolds such as cyclic urea (DMP 450), 4-hydroxy-2-pyrone (tipranavir), in addition to inhibitors of cytokine converting enzymes (TACE (TNF converting enzyme), ICE (interleukin beta converting enzyme)) and related proteases.

Cytostatics and Immune Suppressants

Vitamin D3 derivatives (e.g. calcipotriole, cholecalciferol).

Dignostics

Fluorescent Cell Markers

Bodipy-propionic acid.

Sterol Synthesis Modifying Agents

Atorvastatin, Pravastatin, Simvastatin, Lovastatin, Cerivastatin, Roxuvastatin, Fluvastatin.

Also within the scope of this invention is a pharmaceutical composition that contains an effective amount of at least one of the compound of this present invention and a pharmaceutically acceptable carrier.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, is digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, mesylate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

Further, this invention covers a method of administering an effective amount of one or more compounds of this invention to a subject (a human, a mammal, or an animal) in need of treatment for a disease or disease symptom (e.g., an inflammatory disease, an infectious disease, cancer, allergy, or an immune disease, or symptoms thereof).

The term “treating” or “treated” refers to administering a compound of this invention to a subject with the purpose to cure, heal, alleviate, relieve, alter, remedy, ameliorate, improve, or affect a disease, the symptoms of the disease or the predisposition toward the disease. “An effective amount” refers to an amount of a compound which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 20 mg/Kg. Effective doses will also vary, as recognized by those skilled in the art, depending on route of administration, excipient usage, and the possibility of co-usage with other agents for treating a disease, including an inflammatory disease, a cardiovascular disease, an infectious disease, cancer, allergy, and an immune disease.

The methods delineated herein can also include the step of identifying that the subject is in need of treatment of for a disorders and or condition in athe subject. The identification can be in the judgment of a subject or a health professional and can be subjective (e.g., opinion) or objective (e.g., measurable by a test or a diagnostic method).

The following is a non-exclusive list of diseases and disease symptoms, which may be treated or prevented by administration of the compounds and compositions thereof herein and by the methods herein.

Inflammation and Related Disorders

Inflammation Secondary to Trauma or Injury

Post traumatic regeneration injury including but not limited to Ischemia, reperfusion injury, scarring, CNS trauma, spinal section, edema, repetitive strain injuries including tendonitis, carpal tunnel syndrome,

Cardiovascular Diseases

specifically atherosclerosis, inflamed or unstable plaque associated conditions, restinosis, infarction, thromboses, post-operative coagulative disorders, acute stroke,

Autoimmune Diseases

Alopecia Areata, Ankylosing Spondylitis, Antiphospholipid Syndrome, Autoimmune Addison's Disease, aplastic anemia, Autoimmune Hemolytic Anemia, Autoimmune Hepatitis, Behcet's Disease, biliary cirrhosis, Bullous Pemphigoid, Canavan Disease, Cardiomyopathy, Celiac Sprue-Dermatitis, Chronic Fatigue Immune Dysfunction Syndrome (CFIDS), Chronic Inflammatory Demyelinating Polyneuropathy, Churg-Strauss Syndrome, Cicatricial Pemphigoid, CREST Syndrome, Cold Agglutinin Disease, Crohn's Disease, dermatomyositis, Diffuse Cerebral Sclerosis of Schilder, Discoid Lupus, Essential Mixed Cryoglobulinemia, Fibromyalgia-Fibromyositis, Fuch's heterochromic iridocyclitis, Graves' Disease, Guillain-Barré, Hashimoto's Thyroiditis, Idiopathic Pulmonary Fibrosis, Idiopathic Thrombocytopenia Purpura (ITP), IgA Nephropathy, Insulin dependent Diabetes, Intermediate uveitis, Juvenile Arthritis, Lichen Planus, Lupus, Ménière's Disease, Mixed Connective Tissue Disease, Multiple Sclerosis, Myasthenia Gravis, nephrotic syndrome, Pemphigus Vulgaris, Pernicious Anemia, Polyarteritis Nodosa, Polychondritis, Polyglandular Syndromes, Polymyalgia Rheumatica, Polymyositis and Dermatomyositis, Primary Agammag-lobulinemia, Primary Biliary Cirrhosis, Psoriasis, Raynaud's Phenomenon, Reiter's Syndrome, Rheumatic Fever, Rheumatoid Arthritis, Sarcoidosis, Scleroderma, Sjogren's Syndrome, Stiff-Man Syndrome, Takayasu Arteritis, Temporal Arteritis/Giant Cell Arteritis, Ulcerative Colitis, Vasculitis, Vitiligo, VKH (Vogt-Koyanagi-Harada) disease, Wegener's Granulomatosis, Anti-Phospholipid Antibody Syndrome (Lupus Anticoagulant), Churg-Strauss (Allergic Granulomatosis), Dermatomyositis/Polymyositis, Goodpasture's Syndrome, Interstitial Granulomatous Dermatitis with Arthritis, Lupus Erythematosus (SLE, DLE, SCLE), Mixed Connective Tissue Disease, Relapsing Polychondritis, HLA-B27 asssociated conditions including Ankylosing spondylitis, Psoriasis, Ulcerative colitis, Crohn's disease, IBD, Reiter's syndrome, Uveal diseases: Uveitis, Pediatric Uveitis, HLA-B27 Associated Uveitis, Intermediate Uveitis, Posterior Uveitis, Iritis,

Dermatological Disease

Psoriasis, atopic dermatitis, acne

Rheumatological Disease

Osteoarthritis and various forms of autoimmune arthritis.

Neurodegenerative Disease

Inflammatory Degenerative Diseases

Including variants and major forms of: Alzheimer's, Huntington's Parkinson's and Creutzfeldt Jakob disease

Infection

Respiratory diseases of diverse origin including:

Pharyngitis (“sore throat”), Tonsilitis, Sinusitis & Otitis Media, Influenza, Laryngo-Tracheo Bronchitis (Croup), Acute Bronchiolitis, Pneumonia, Bronchopneumonia, Severe Acute Respiratory Syndrome (SARS), Bronchiolitis, Bronchitis, Acute pharyngitis with fever, Pharyngoconjunctival fever, Acute follicular conjunctivitis, Pneumonia (and pneumonitis in children), COPD, asthma,

Gastrointestinal Diseases

Gastroenteritis of diverse origin

Viral Diseases

Target viuses include but are not limited to: Paramyxo-, Picoma-, rhino-, coxsackie-, Influenza-, Herpes-, adeno-, parainfluenza-, respiratory syncytial-, echo-, corona-, Epstein-Barr-, Cytomegalo-, Varicella zoster, Hepatitis variants including hepatitis C Virus (HCV), Hepatitis A Virus (HAV), Hepatitis B Virus (HBV), Hepatitis D Virus (HDV), Hepatitis E Virus (HEV), Hepatitis F Virus (HFV), Hepatitis G Virus (HGV), Human immunodeficiency-

Neoplastic Disease

leukemia, lymphoma, myeloma

hepatomas, other major organ carcinomas and sarcomas

glioma, neuroblastoma,

Astrocytic and glial tumors,

Invasive or non-invasive (Anaplastic (malignant) astrocytoma, Glioblastoma multiforme variants: giant cell glioblastoma, gliosarcoma, Pilocytic astrocytoma, Subependymal giant cell astrocytoma, Pleomorphic xanthoastrocytoma)

Oligodendroglial Tumors

Ependymal cell tumors, Mixed gliomas, Neuroepithelial tumors of uncertain origin, Tumors of the choroid plexus, Neuronal and mixed neuronal-glial tumors, Pineal Parenchyma Tumors, Tumors with neuroblastic or glioblastic elements (embryonal tumors), Neuroblastoma, ganglioneuroblastoma, Tumors of the Sellar Region, Hematopoietic tumors, Primary malignant lymphomas, Plasmacytoma, Granulocytic sarcoma, Germ Cell Tumors, Tumors of the Meninges

Allergy

Rhinitis, bronchitis, asthma and conditions relating to excessively active or stimulated eosinophils.

Transplant Medicine

Renal, hepatic, corneal, stem cell, pulmonary, cardiac, vascular, and myeloid transplants

Metabolic Disease,

Various disorders clustered in the liver cirrhosis, dyslipidemia, diabetes, obesity, coagulation disorders, and hypercholesterolemia groupings.

### BENEFITS OF THE INVENTION

The conjugates described here represent improvements on their parent therapeutic agents in two main respects. First, these conjugates provide a facile means of improving the activity of a therapeutic agent through their ability to make the therapeutic agent more easily available either from the gut, or from the blood stream. This is especially important for those therapeutic agents that have good activity in vitro but are unable to exert that activity in vivo. Where the non-manifestation of activity is related to inefficient uptake and distribution, simple conjugations according to the schemes described here are an efficient means to generate improved activity.

The invention also has specific benefits. By targeting cells, and achieving higher concentration in those cells than in plasma or general tissue, the therapeutic agent may exert a more specific action resulting in fewer systemic side effects. Where efficacy is limited by the ability to place sufficient therapeutic agent at the site of action, such concentration effects are significant in achieving improved in vitro effect. This may be understood more clearly by examination of non-limiting but representative examples from different therapeutic areas.

In Example 2 or 8, improved anti-inflammatory therapeutic agents are described in which the active molecules are concentrated into immune cells in vitro through conjugation with a macrolide. These conjugates display superior immune suppressive and anti-inflammatory action in vivo when compared with the effect of a mixture of the two component molecules in the same system. The mechanism for this action is unknown but the effect in protection appears to be qualitatively similar for the mixture and the conjugate suggesting that the conjugate is largely a delivery mechanism for the therapeutic agent. The conjugate also has other potential benefits including the prevention of metabolism through steric effects, increased residence time and traffic to sites of inflammation when it is taken up into target cells which are tropic for the inflamed tissues. Some action of the conjugate itself cannot be ruled out when it is present at high concentrations in a cell.

In Examples 3 and 4, an anti-viral therapeutic agent conjugate is cited that also achieves higher levels in immune cells which may act as a reservoir of integrated viral material. If therapeutic agent is selectively conjugated such that it is concentrated in these cells, it has two potential benefits including, the ability to suppress viral replication at lower systemic doses, and the ability to prevent resistance through the maintenance of persistently higher concentrations of therapeutic agent such that mutations with minor effect cannot accumulate.

Similar themes but contrasting mechanisms apply to the field of graft rejection where one focus of therapy is the prevention of T-cell responses to the donor organ. Various mechanisms are known but all would benefit if a greater proportion of chemical effect were focused on the T-cells themselves such that the systemic dose were reduced. Example 6 cites conjugates of vitamin D analogs that are highly concentrated in immune cells.

Data to support these observations may be found in various examples and is summarized here by reference in a non-limiting manner.

To practice the method of treating a disease, the compounds of this invention can be administered to a patient, for example, in order to treat a disease described above. The compound can, for example, be administered in a pharmaceutically acceptable carrier such as physiological saline, in combination with other therapeutic agents, and/or together with appropriate excipients. The compound described herein can, for example, be administered by injection, intravenously, intraarterially, subdermally, intraperitoneally, intramuscularly, or subcutaneously; or orally, buccally, nasally, transmucosally, topically, in an ophthalmic preparation, by inhalation, by intracranial injection or infusion techniques, with a dosage ranging from about 0.1 to about 20 mg/kg of body weight, preferably dosages between 10 mg and 1000 mg/dose, every 4 to 120 hours, or according to the requirements of the particular therapeutic agent. The methods herein contemplate administration of an effective amount of compound or compound composition to achieve the desired or stated effect. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, therapeutic agent combination, the severity and course of the disease, condition or symptoms, the patient's disposition to the disease, condition or symptoms, and the judgment of the treating physician.

Pharmaceutical compositions of this invention comprise a compound of this invention or a pharmaceutically acceptable salt thereof; and any pharmaceutically acceptable carrier, adjuvant or vehicle. Such compositions may optionally comprise additional therapeutic agents. The compositions delineated herein include the compounds of the formulae delineated herein, as well as additional therapeutic agents if present, in amounts effective for achieving a modulation of a disease.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying therapeutic agent delivery systems (SEDDS) such as D-alpha-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein. Oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A suitable in vitro assay can be used to preliminarily evaluate a compound of this invention in treating a disease. In vivo screening can also be performed by following procedures well known in the art. See the specific examples below.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will be further described in the following example. It should be understood that these examples are for illustrative purposes only and are not to be construed as limiting this invention in any manner.

### EXAMPLES

| Example number | Subject |
|---|---|
| 1. | Method for determining immune cell partition |
| 2. | Simvastatin conjugate |
| 3. | Indinavir conjugate |
| 4. | Indinavir conjugate |
| 5. | Amprenavir conjugate |
| 6. | Cholecalciferol conjugate |
| 7. | BODIPY conjugate |
| 8. | Genistein conjugate |
| 9. | Genistein conjugate |
| 10. | FACS based uptake determinaiton |

### Example 1

### Determination of Drug Uptake

Uptake of Compounds

Freshly drawn heparinised blood or buffy coat preparations are used for the determination of immune cell partition ratios. Buffy coat preparations are preferred. These may be obtained from donor blood by simple centrifugation of whole blood (4795 g for 10 minutes). Following centrifugation, plasma is collected from the surface, after which immune cells are expressed from the donor bags along with the erythrocytes lying immediately below the leukocyte layer. This ensures high yields and a sufficient population of erythrocytes for partition. 5 ml of the resulting cell suspension are dispensed into T25 culture flasks. Substrates are added to a final concentration between 1 and 10 μM and the suspensions incubated at 37° C., in a 5% CO₂ atmosphere. For analysis of uptake kinetics, samples are withdrawn at 0, 2, 5, 10, 30, 60, 90, 180, or 240 min after substrate addition. For screening purposes, samples are taken at 0 and 120 minutes.

Buffers and solutions

| | |
|---|---|
| PBS | 73 mM NaCl, 2.7 mM KCl, 1.5 mM KH₂PO₄, |
| | 8 mM Na₂HPO₄, pH 7.4 |
| DPBS | 137 mM NaCl, 3 mM KCl, 8 mM Na₂HPO₄, |
| | 1 mM KH₂PO₄, 1 mM CaCl₂, 0.5 mM |
| | MgCl₂, 5 mM Glucose, pH 7.4 |

Separation of Blood Cell Fractions—Density Gradient Centrifugation

Cell fractions were prepared using density gradient centrifugation. Mononuclear cells and polymorphonuclear cells are separated from erythrocytes essentially by layering the cell suspension on a viscous medium typically composed of a solution containing Ficoll or similar (commercial suppliers include: Lymphoprep, Opti-prep from Axis Shield, 1031966; Lymphoflot HLA, 824010; or PMN Separation Medium Robbins Scientific 1068-00-0). The layered suspension is then centrifuged at 600 g, 20 min, after which the cell fractions and the plasma (incubation medium) fraction are removed by gentle aspiration, washed twice in PBS buffer, followed by estimation of the cell number and pellet volume.

Analysis

Uptake of fluorescent compounds is monitored using fluorescence microscopy. Excitation and emission wavelengths depend of the fluorescence label in use. A typical label is a methoxy coumarin for which the appropriate wavelengths are 360 and 450 nm respectively. Fluorescent analogs of the compounds under study permit the estimation of appropriate uptake intervals as well as the likely intracellular distribution of the compounds. Fluorescent analogs also allow the estimation of losses in washing or other cell manipulations.

Cell preparations are lysed in water and the debris sedimented at 16100 g, 10 min. The supernatant is recovered and sub-sampled for protein and DNA content. Protein in the supernatant is precipitated by bringing the solution to 80-100% v/v acetonitrile and centrifuging again at 16100 g, 10 min.

Compound uptake is normalized according to cytoplasmic volume of cells in order to obtain the average concentration in the cells. Cell volume is estimated by correlation of DNA, protein or haem content of lysed cell aliquots to cell number and packed volume prior to lysis.

Cell lysates are analysed using a HP 1100 HPLC System (Agilent Technologies, Waldbronn, Germany) with a Kromasil 3.5μ C18, 50×2.0 mm column and guard cartridge system (both, Phenomenex, Aschaffenburg, Germany) run at 30° C. A gradient elution was performed using water, 0.05% formic acid (A) and acetonitrile 0.05% formic acid (B) (0 min. 5% B, 2.5 min 5% B, 2.8 min 40% B, 10.5 min 85% B, 12.0 min 95% B, 16.5 min 95% B) at a flow rate of 300 μl/min. Re-equilibration of column was at 5% B, at a flow rate of 750 μl/min for 2.4 min. The HPLC-eluate from retention time 0.0 min to 2.5 min was directed directly to waste. Detection was via a UV cell at 214 run followed by a 1/6 split to an An API-qTOF 1 (Micromass, Manchester, UK) mass spectrometer, (calibrated daily using a mixture of NaI, RbI and CsI). The mass spectrometer is routinely operated in the positive electrospray ionization mode using the following settings: Capillary voltage 4000 V; cone voltage 30 V; RF Lens offset 0.38 V; source block temperature 80° C.; desolvation gas temperature 140° C.; desolvation gas 240 l/h; LM/HM Resolution 0.0; Collision energy 4.0 V; Ion energy 5.0 V.

Masses are monitored according to the known or expected M/Z ratios. Ion currents across the expected range of masses (including metabolites) are recorded and the chromatograms for specific masses used to estimate the peak area for a given molecular ion (area proportional to concentration over a given range). Normalisation to DNA and/or protein and/or haem content of cells (all three measured with standard methods (Bisbenzimide staining (Sigma), BCA protein assay kit (Pierce) and haem absorbance at 535 nm, respectively)) to cell number (hemocytometer or FACS count) and cell volume is employed to calculate average compound concentration in the cell fraction (expressed in uM). Formation of metabolites or hydrolysis products was also monitored for each T-L-C conjugate and the rate of hydrolysis estimated from both the total uptake and the loss of metabolites to the medium. The final ratio is computed by comparing the concentration of a component in the immune cell compartment with that in both the erythrocytes and the plasma. The P_{ISR}, is then the concentration in immune cells/concentration in erythrocytes using the same concentration units. Thus a P_{ISR} of 2 indicates a two-fold concentration relative to erythrocytes.

| | | Erythrocytes/plasma | |
|---|---|---|---|
| Compound | MNCs (μM) | (μM) | Ratio |
| 1 | 14.50 | 7.10 | 2.04 |
| 2 | 8.84 | 1.09 | 8.90 |
| 4 | 4.11 | 0.21 | 19.57 |
| 9 | 11.64 | 1.03 | 11.25 |
| 10 | 10.34 | 0.19 | 54.98 |

### Example 2

### Compound 4

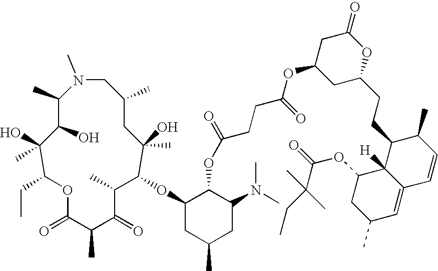

A solution of 420 mg of simvastatin in 3 ml of dichloromethane was treated with 110 mg of succinic anhydride and 10 mg of DMAP. After 36 h, 210 mg of EDCI and 600 mg of Compound 2 was added under stirring. After 1 h, the mixture was passed through a pad of silica gel, eluting with chloroform:isopropanol:methanolic ammonia (30:1:1) to yield Compound 4 as an off white solid (440 mg; 40% yield). TLC: R_{f} 0.38 (chloroform:isopropanol:methanolic ammonia (30:1:1)). MS: M⁺ 1090.

### Example 3

### Compound 5

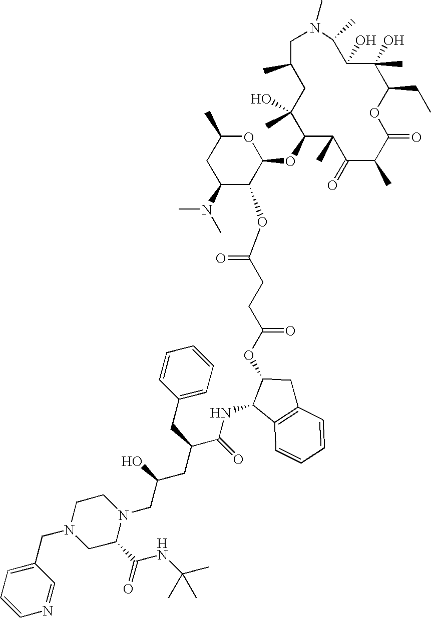

A solution of 850 mg of indinavir in 5 ml of dichloromethane was treated with 152 mg of succinic anhydride and 34 mg of DMAP. After 36 h, 300 mg of EDCI and 585 mg of Compound 2 was added under stirring. The reaction mixture was stirred overnight at room temperature. At this point the mixture was concentrated in vacuo and passed through a pad of silica gel, eluting with chloroform:isopropanol:methanolic ammonia (30:1:1) to yield Compound 5 as an off white foam (500 mg; 30% yield). TLC: R_{f} 0.54 (chloroform:isopropanol:methanolic ammonia (30:1:1)). MS: M⁺ 1284.

### Example 4

### Compound 6

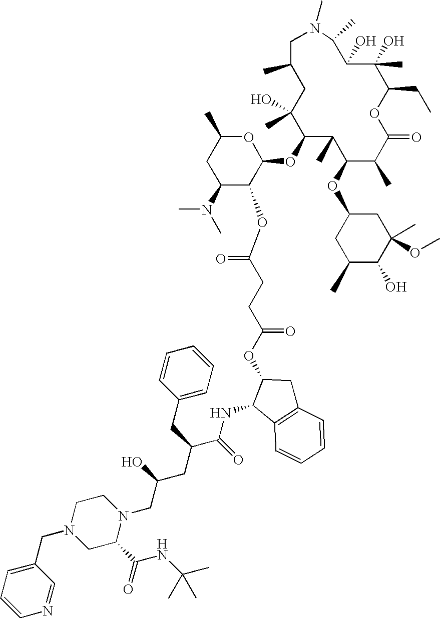

Compound 1 (749 mg, 1 mmole) was treated with succinic anhydride (1.2 eq, 120 mg, 1.2 mmole) in presence of catalytic amount of DMAP (10 mg) in dry DCM (2.5 mL) for 2 days at room temperature under argon. To 0.2 ml (0.08 mmole) of the previous reaction mixture were added Indinavir (1.1 eq, 0.1 mmole, 61 mg) and EDCI (1.5 eq, 0.15 mmole, 30 mg). The mixture was stirred at room temperature over night. The expected compound was isolated and purified by column chromatography using silica gel (200 g), Chloroform/Methanol/Ammonia(7N in Methanol) (850:40:40) as the eluent. The collected fractions were concentrated to yield Compound 6 as a slightly yellowish solid, and indinavir as an impurity (total 70 mg). The product was characterized by MS MH⁺ 1444.5 and TLC R_{f} 0.3 in CMA(850:40:40).

### Example 5

### Compound 7

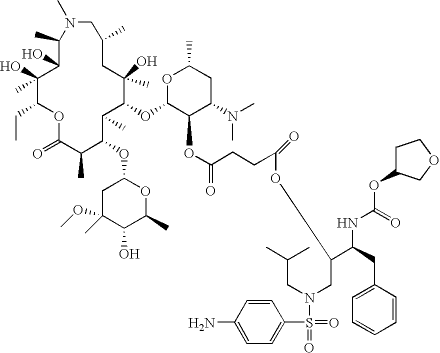

Compound 1 (749 mg, 1 mmol, 1 eq) was dissolved in 1 ml of THF (dried over molecular sieve) and added to a solution of succinic anhydride (120 mg, 1.2 mmol, 1.2 eq) in 1 ml of THF (dried over molecular sieves) in the presence of DMAP (12 mg, 0.1 mmol, 0.1 eq). The clear colorless solution was stirred 24 h at room temperature under argon. The reaction was checked for completion by TLC (Chloroform: Isopropanol: Ammonia 7N in methanol=30:1:1) by consumption of compound 1. The solution was concentrated to 1 ml and taken into the next step without any further purification. The solution above was mixed with a solution (dried over molecular sieves) of amprenavir (162 mg, 0.32 mmol), Py-BOP (433 mg, 0.80 mmol) and N,N-diisopropylethylamine (177 μl, 0.96 mmol) in 1 ml of THF. The clear colorless solution was allowed to stir at RT under argon 12 h. Evaporation of the solvent yielded a white-yellowish foam that was purified by column chromatography (Chloroform: Isopropanol: Ammonia 7N in methanol=30:1:1). The collected fractions were concentrated to give Compound 7 as a yellowish foam (128 mg, 30%). The product was characterized by MS (MH+: 1336) and TLC (Rf: 0.4 solvent system: Chloroform: Isopropanol: Ammonia 7N in methanol=30:1:1).

### Example 6

### Compound 8

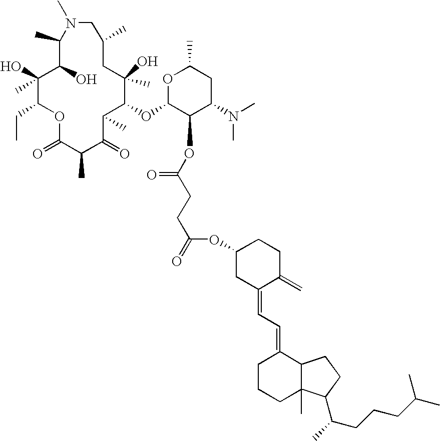

A solution of 380 mg of cholecalciferol in 3 ml of dichloromethane was treated with 100 mg of succinic anhydride and 12 mg of DMAP. After 72 h, 250 mg of EDCI and 590 mg of compound 2 was added under stirring (compound 3 may also be used). The reaction mixture was stirred overnight at room temperature. At this point the mixture was concentrated in vacuo and passed through a pad of silica gel, eluting with chloroform:isopropanol:methanolic ammonia (30:1:1) to yield Compound 8 as a yellowish foam (264 mg; 25% yield). TLC: R_{f} 0.30 (chloroform:isopropanol:methanolic ammonia (30:1:1)). MS: M⁺ 1055.

### Example 7

### Compound 9

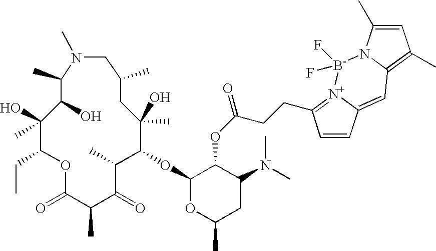

A solution of 500 mg of bodipy-propionic acid and 25 mg of DMAP in 10 ml of dichloromethane was treated with 489 mg of EDCI at room temperature. After 5 min, Compound 2 was added under stirring. The reaction mixture was stirred under Ar overnight at room temperature. At this point the mixture was concentrated in vacuo. Column chromatography of the resulting residue (silica gel, eluting solvent chloroform:isopropanol:methanolic ammonia (30:1:1)) gave Compound 9 as a white foam (366 mg; 25% yield). TLC: R_{f} 0.4 (chloroform:isopropanol:methanolic ammonia (30:1:1)). MS: M⁺ 863.

### Example 8

### Compound 10

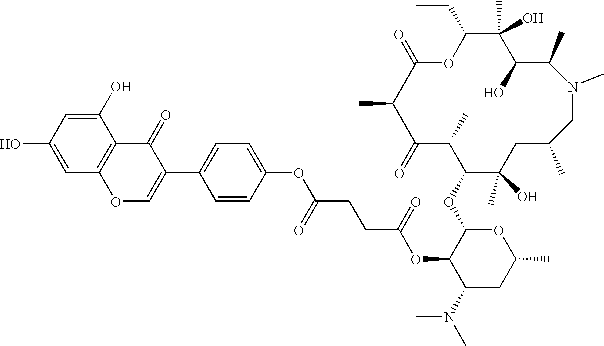

A solution of 150 mg of Compound 2 in 2 ml of THF was treated with 31 mg of succinic anhydride and 53 μL of DIEA. The reaction was stirred overnight at room temperature, and then it was cooled to 0° C. under Ar. To this, 84 mg of N,N-dicarbonylimidazole was added. After 30 min, 102 mg genistein was added. The reaction was once again stirred overnight at room temperature. The solvent was removed in vacuo. The residue was taken up in EtOAc and acid-base extraction ensued providing a off-white solid, Compound 10 (95 mg; 40% yield). TLC: R_{f} 0.16 (chloroform:isopropanol:methanolic ammonia (30:1:1)). MS: M⁺ 941.

### Example 9

### Compound 11

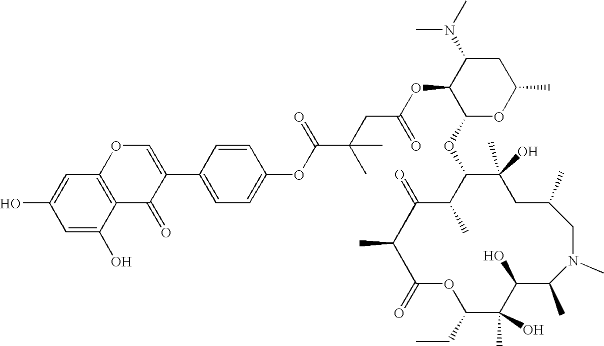

A solution of 150 mg of Compound 2 in 2 ml of THF was treated with 40 mg of 2,2-dimethylsuccinic anhydride and 3 mg of 4-DMAP. The reaction was stirred overnight at room temperature, at which point, the reaction was cooled to 0° C. under Ar. To this, 87 mg of N,N-dicarbonylimidazole was added. After 30 min, 102 mg genistein was added. The reaction was once again stirred overnight at room temperature. The solvent was removed in vacuo. The residue was taken up in EtOAc and acid-base extraction ensued providing Compound 11 as a yellow solid (98 mg; 42% yield). TLC: R_{f} 0.15 (chloroform:isopropanol:methanolic ammonia (30:1:1)). MS: M⁺ 969.

### Example 10

### FACS Based Uptake Assay

Freshly collected mouse whole blood was incubated in the presence of compound 9 or BODIPY propionic acid. After 60 minutes, the mixture was subject to fluorescent cytometry using a MoFlo fluorescence activated cell sorter (DakoCytomation In.) with detection via a 488 nm Laser. Cells were analysed based on forward and side scatter (which provides a measure of size and granularity) as well as fluorescence intensity. Compound 9 labeled a distinct population of cells more intensively than the organic acid precursor fluophore from which it is derived.

[image]

### OTHER EMBODIMENTS

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### CITATIONS

Alberola A., Antolin L. F., Gonzalez A. M., Laguna M. A., and Pulido F. J.; J. Heterocyclic Chem; 23; 1035; (1986).

Alberola A., Antolin L. F., Gonzalez A. M., Pulido F. J.; Base-induced Ring Cleavage of 4-Functionalized 3-Unsubstituted Isoxazoles. Synthesis of 2-Aminopyrimidines and Pyrimidine-2(3H)-thiones; Heterocycles; (1987).

Antoniou E A, Xu M, Howie A, Chondros K, McMaster P, D'Silva M, 1997 Combination treatment effectively intercepts advanced acute cardiac rejection, Transplant Proc November; 29(7):2888-91.

Arion D, Meijer L, Brizuela L, Beach D, 1988, Cell 55, 371-378.

Axton et al., 1992, J. Chem. Soc. Perkin Trans. 12203 ff.

Bartlett et al., 1991, Agents and Actions, 32 10-21.

Beeson J M 1999, The neurobiology of pain, Lancet;353:1610-1615.

Bennett J E, 1977, Flucytosine. Ann. Intern. Med. 86,319-322.

Benslay D N and Bendele A M, 1991, Agents Actions 34: 254.

Billingham et al., 1954. Proc. R. Soc. 143: 43-55.

Brater D C, 1999, Effects of nonsteroidal anti-inflammatory therapeutic agents on renal function: focus on cyclooxygenase-2-selective inhibition, Am J Med December 13;107(6A):65S-70S; discussion 70S-71S.

Breedveld F C, Dayer J M, 2000, Leflunomide: mode of action in the treatment of rheumatoid arthritis, Ann Rheum Dis November;59(11):841-9.

Buchdunger E, Mett H, Trinks U, Regenass U, Muller M, Meyer T, Beilstein P, Wirz B, Schneider P, Traxler P, 1994, 4,5-Dianilinophthalimide: A protein-tyrosine kinase inhibitor with selectivity to the epidermal growth factor receptor signal transduction pathway and potent in vivo antitumor activity. Proc. Nat. Acad. Sci. USA 91, 2334-2338.

Buchdunger E, Zimmermann J, Mett H, Meyer T, Muller M, Regenass U, Lydon N B, 1995, Selective inhibition of the platelet-derived growth factor signal transduction pathway by a protein-tyrosine kinase inhibitor of the 2-phenylaminopyrimidine class. Proc. Nat. Acad. Sci. USA 92, 2558-2562.

Carlson R P, Datko Li, 0 Neil-Davis L, Blazek E M, Delustro F, Beideman R., and Lewis A J, 1985, Comparison of inflammatory changes in established type II collagen and adjuvant induced arthritis using outbred Wistar rats, J. Immunopharmacol. 7: 811-826.

Churchill L, Graham A G, Shih C-K, Pauletti D, Farina P R, Grob P M, 1996, Selective inhibition of human cyclo-oxygenase-2 by meloxicam. Inflammopharmacology; 4: 125-135.

Colville-Nash P R and Gilroy D W, 2001, Potential adverse effects of cyclooxygenase-2 inhibition: evidence from animal models of inflammation BioTherapeutic agents; 15(1): 1-9.

Conner E M, Grisharn M B, 1996, Inflammation, free radicals and antioxidants, Nutrition 12(4), 274-277.

Cronstein B N, 1995, The antirheumatic agents sulphasalazine and methotrexate share an anti-inflammatory mechanism, Br J Rheumatol November;34 Suppl 2:30-2.

Curkovic B et al., 2000 Nonsteroidal antirheumatic agents—present status and perspectives, Reumatizam;47(2):7-10.

Daley, G Q. & Baltimore, O, 1988 Transformation of an interleukin 3-dependent hematopoietic cell line by the chronic myelogenous leukemia-specific p21Obcr-abl protein. Proc. Nat. Acad. Sci. USA 85, 9312-9316.

De Clercq, E, 1993: Antiviral agents: characteristic activity spectrum depending on the molecular target with which they interact. Academic Press, Inc., New York, N.Y. 1-55

Debruyne D and Ryckelynck J P, 1993, Clinical pharmacokinetics of fluconazole. Clin. Pharmacokinet. 24,10-27.

Debs R J, Fuchs H J, Philip R, Brunette E N, Duzgunes N, Shellito J E, Liggitt D, Patton J R, 1990, Immunomodulatory and toxic effects of free and liposome-encapsulated tumor necrosis factor alpha in rats. Cancer Res. January 15; 50(2):375-80.

Escola J M, Deleuil F, Stang E, Boretto J, Chavrier P, Gorvel J P. Characterization of a lysozyme-major histocompatibility complex class II molecule-loading compartment as a specialized recycling endosome in murine B lymphocytes. J Biol. Chem. 1996 November 1 ;271(44):27360-5.

Ford C W, Hamel J C, Wilson D M, Moerman J K, Stapert D, Yancey R, Hutchinson D K, Barbachyn M R, Brickner S J, 1996, In vivo Activities of U-100592 and U-100766, Novel Oxazlidinone Antimicrobial Agents, against Experimental Bacterial Infections. Animicrobial Agents and Chemotherapy 1508-1513.

Fox R I J, 1998, Mechanism of action of leflunomide in rheumatoid arthritis, Rheumatol Suppl July; 53:20-6.

Fung H B, Kirschenbaum H L, 1999, 57Selective cyclooxygenase-2 inhibitors for the treatment of arthritis, Clin Ther July;21(7):1131-1135.

Furst D E, 1999, Leflunomide, mycophenolic acid and matrix metalloproteinase inhibitors, Rheumatology (Oxford) November; 38 Suppl 2:14-8.

Gilbert, B. E. and Knight V., 1986: Biochemistry and clinical applications of ribavirin. Antimicrob. Agents and Chemother. 30:201-205

Gull K and Trinci A P J, 1973, Griseofulvin inhibits fungal mitosis. Nature. 244, 292-294.

Harada M, Sakakibara H, Yano T, Suzuki T, Okuno S, 2000, Determinants for the therapeutic agent release from T-0128, camptothecin analogue-carboxymethyl dextran conjugate. J Control Release. December 3;69(3):399-412.

Hata K, Kimura J, Miki H, Toyosawa T, Moriyama M and Katsu K, 1996, Efficacy of ER-30346, a novel oral triazole antifungal agent, in experimental models of Aspergillosis, Candidiasis and Cryptococcosis. Antimicrobial Agents and Chemotherapy, 40,2243-2247.

Hawkey C J, 1999, COX-2 inhibitors. Lancet; 353:307-314.

Hepatology 7: 724-731

Hial V, De Mello M C, Horakova Z, Beaven M A, 1977, Antiproliferative activity of anti-inflammatory therapeutic agents in two mammalian cell culture lines J Pharmacol Exp Ther. August;202(2):446-54.

Honda S, Migita K, Hirai Y, Origuchi T, Yamasaki S, Kamachi M, Shibatomi K, Fukuda T, Kita M, Hida A, Ida H, Aoyagi T, Kawakami A, Kawabe Y, Oizumi K, Eguchi K, 2001, Expression of membrane-type 1 matrix metalloproteinase in rheumatoid synovial cells, Clin Exp Immunol 2001 October;126(1):131-6.

Hultgren, C., Millich, D. R, Weiland, O., Salberg, M., 1998: The antiviral compound ribavirin modulates the T-helper (Th)1/Th2 subset balance in hepatitis B and C virus-specific immune response. J. Gen. Virol. 79: 2381-2391

Hutchins R O, Hoke D, Keogh J, Koharstki D, 1969, Sodium Borohydride in Dimethyl Sulfoxide or Sulfolane. Convenient Systems for Selective Redutions of Primary, Secondary, and Certain Tertiary Halides and Tosylates. Tetrahedron Letters, 3495-3498.

Iñliguez M A, Punzón C, Fresno M, 1999, Induction of Cyclooxygenase-2 on Activated T Lymphocytes: Regulation of T Cell Activation by Cyclooxygenase-2 Inhibitors, The Journal of Immunology, 163: 111-119.

Jaafari M R, Foldvari M, 2002, Targeting of Liposomes to Human Keratinocytes Through Adhesive Peptides from Immunoglobulin Domains in the Presence of IFN Therapeutic agent Deliv. 9(1):1-9.

Kalgutkar A S, Crews B C, Rowlinson S W, Marnett A B, Kozak K R, Remmel R P, Marnett L J, 2000, Biochemically based design of cyclooxygenase-2 (COX-2) inhibitors: facile conversion of nonsteroidal antiinflammatory therapeutic agents to potent and highly selective COX-2 inhibitors, Proc Natl Acad Sci USA January 18;97(2):925-30.

Kanakura Y, Druker B, Cannistra S A, Furukawa Y, Torimoto Y, Griffin J D, 1990, Signal transduction of the human granulocyte-macrophage colony-stimulating factor and interleukin-3 receptors involves a common set of cytoplasmic proteins. Blood 76, 706-715.

Kharbanda S, Ren R, Pandey P, Shafman T D, Feller S M, Weichselbaum R R, Kufe D W, 1995, Activation of the c-abl tyrosine kinase in stress response to DNA-damaging agents. Nature 376, 785-788.

Klegeris A, McGeer P L, 1994, Rat brain microglia and peritoneal macrophages show similar responses to respiratory burst stimulants, Journal of Neuroimmunology 53(1), 83-90.

Klegeris A, Walker D G, McGeer P L, 1994, Activation of macrophages by Alzheimer b amyloid peptide, Biochemical and Biophysical Research Communications 199(2), 984-991.

Korba, B. E. and Boy, M. R, 1996: penciclovir is a selective inhibitor of hepatitis B replication in cultured human hepatoblastoma cells. Antimicrob. Agents and Chemother. 40:1282-1284

Kurtz M B, Heath I B, Marrinan J, Dreikorn S, Onishi J and Douglas C, 1994, Morphological effects of lipopeptides against Aspergillus fumigatus correlate with activities against (1,3)-b-D-glucan synthase. Antimicrob. Agents Chemother. 38,1480-1489.

Labro M T and Abdelghaffar H, 2001, Immunomodulation by macrolide antibiotics. J. Chemother. February;13(1):3-8. Review.

Lipinski C A, Lombardo F, Dominy B W, Feeney P J., Experimental and computational approaches to estimate solubility and permeability in therapeutic agent discovery and development settings., Adv Therapeutic agent Deliv Rev. 2001 March 1 ;46(1-3):3-26. Review.

Malmstrom K, Daniels S, Kotey P, Seidenberg B C, Desjardins P J, 1999, Comparison of rofecoxib and celecoxib, two cyclooxygenase-2 inhibitors, in postoperative dental pain: a randomized, placebo- and active-comparator-controlled clinical trial, Clin Ther October;21(10):1653-63.

Marion, P. L., Cullen, J. M., Azcarraga, R. R., Van Davelaar, M. J., Robinson, W. S., 1987: Experimental transmission of duck hepatitis B virus to pekin ducks and to domstic geese.

Marks D, Belov L, Davey M W, Davey R A, Kidman A, 1992, The MTT cell viability assay for cytotoxicity testing in multitherapeutic agent-resistant human leukemic cells, Leukemia Research 16, 1165.

Marriott M S and Richardson K, 1987, The discovery and mode of action of fluconazole, p. 81-92. In R. A. Fromtling (ed.), Recent trends in the discovery, development, and evaluation of antifungal agents. J. R. Prous Science Publishers, Barcelona.

Martin, M. J., Navas, S., Quiroga, J. A., Pardo, M., Carreno, V., 1998: Effects of the ribavirin-interferon alpha combination on cultured peripheral blood mononuclear cells from chronic hepatitis C patients. Cytokine August;10(8):635-44.

Martin, M. J., Navas, S., Quiroga, J. A., Pardo, M., Carreno, V., 1998: Effects of the ribavirin-interferon alpha combination on cultured peripheral blood mononuclear cells from chronic hepatitis C patients. Cytokine August;10(8):635-44.

Martin, Pipasha Biswas, Mann, 2000, The incidence of adverse events and risk factors for upper gastrointestinal disorders associated with meloxicam use amongst 19 087 patients in general practice in England: cohort study, British Journal of Clinical Pharmacology 50 Issue 135.

Matsuguchi T, Salgia R, Hallek M, Eder M, Druker B, Ernst T J, Griffin J D, SHC phosphorylation in myeloid cells is regulated by GM-CSF, IL-3, and steel factor and is constitutively increased by p2IOBCR-ABL. J. Biol. Chem. 269, 5016-5021.

Matulonis U, Salgia R, Okuda K, Druker B, Griffin J D, 1993, IL-3 and p21 OBCR/ABL activate both unique and overlapping pathways of signal transduction in a factor-dependent myeloid cell line. Exp. Hematol. 21, 1460-1466.

McGeer E G, McGeer P L, 1997, Inflammatory cytokines in the CNS, CNS Therapeutic agents 7, 214-228.

McGeer P L, McGeer E G, 1995, The inflammatory system of brain: implications for therapy of Alzheimer and other neurodegenerative disorders, Brain Research Review 21(2),195-218.

McGeer P L, Rogers J., 1992, Anti-inflammatory agents as a therapeutic approach to Alzheimer's disease, Neurology 42(2), 447-449.

McGeer P L, Schulzer M, McGeer E G, 1996, Arthritis and antiinflammatory agents as negative risk factors for Alzheimer disease: A review of seventeen epidemiological studies, Neurology 47(2), 425-432.

Mochitate K, Katagiri K, and Miura T, 2001, Impairment of microbial killing and superoxide-producing activities of alveolar macrophages by a low level of ozone, J. Health Science, 47,302-309.

Mortellaro A, Songia S, Gnocchi P, Ferrari M, Formasiero C, D'Alessio R, Isetta A, Colotta F, Golay J, 1999, New immunosuppressive therapeutic agent PNU156804 blocks IL-2-dependent proliferation and NF-kB and AP-1 activation. The Journal of Immunology, 7102-7109.

Nielsen O H, Bukhave K, Elmgreen J, Ahnfelt-Ronne I., 1987, Inhibition of 5-lipoxygenase pathway of arachidonic acid metabolism in human neutrophils by sulfasalazine and 5-aminosalicylic acid, Dig Dis Sci Jun;32(6):577-82.

Pendergast A M, Muller A J, Havlik M H, Clark R, McCormick F, Witte O N, 1991, Evidence for regulation of the human ABL tyrosine kinase by a cellular inhibitor. Proc. Nat. Acad. Sci. USA 88, 5927-5931.

Penglis P S, James M J, Cleland L G, 200, 1Cyclooxygenase inhibitors: any reservations?, InternMedJ January-February;31(1):37-41.

Quallich L G, Greenson J, Haftel H M, Fontana R J, 2001, Is it Crohn's disease? A severe systemic granulomatous reaction to sulfasalazine in patient with rheumatoid arthritis, BMC Gastroenterol;1(1):8.

Rainsford K D, 2001, The ever-emerging anti-inflammatories. Have there been any real advances? J Physiol Paris January-December;95(1-6):11-9.

Ramasamy, K. S., Tam, R. C., Bard J, Averett, D. R., 2000: Monocyclic L-nucleosides with type I cytokine-inducing activity. J Med Chem. 2000 Mar. 9;43(5):1019-28.

Rankin, J. T., Eppes, S. B., Antczak, J. B., Joklik, W. K, 1989: Studies on the mechanism of the antiviral activity of ribavirin against reovirus. Virology 168: 147-158

Roher A E, Chaney M O, Kuo Y M, et al., 1996, Morophology and toxicity of A-beta (1-42) derived from neuritic and vascular amyloid deposits of Alzheimer's disease, Journal of Biological Chemistry 271(34), 20631-20635.

Rossi L, Brandi G, Fraternale A, Schiavano G F, Chiarantini L, Magnani M., Inhibition of murine retrovirus-induced immunodeficiency disease by dideoxycytidine and dideoxycytidine 5′-triphosphate., J Acquir Immune Defic Syndr. 1993 November;6(11):1179-86.

Schindler T, Bornmann W, Pellicena P, Miller W T, Clarkson B, Kuriyan J, 2000, Structural Mechanism for STI-571 Inhibition of Abelson Tyrosine Kinase, Science 289, 1938-1942.

Schluesener H J, Seid K, Deininger M, Schwab J, 2001, Transient in vivo activation of rat brain macrophages/microglial cells and astrocytes by immunostimulatory multiple CpG, oligonucleotides., J Neuroimmunol. February 1 ;113(1):89-94.

Schrier D J, Moniot S, Gluckman M I, Gilbertsen R B, 1987, The topical anti-inflammatory effects of a topical preparation of meclofenamic acid on carrageenan-induced footpad swelling in mice. J Pharm Pharmacol January;39(1):57-9

Schroit A J, Madsen J, Nayar R, 1986, Liposome-cell interactions: in vitro discrimination of uptake mechanism and in vivo targeting strategies to mononuclear phagocytes., Chem Phys Lipids. June-July; 40(2-4):373-93.

Schwab J M, Brechtel K, Nguyen T D, Schluesener H J., Persistent accumulation of cyclooxygenase I (COX-1) expressing microglia/macrophages and upregulation by endothelium following, spinal cord injury., J Neuroimmunol. 2000 Nov. 1; 111(1-2):122-30.

Schwab J M, Frei E, Klusman I, Schnell L, Schwab M E, Schluesener H J, 2001, AIF-1 expression defines a proliferating and alert microglial/macrophage phenotype following spinal cord injury in rats., J Neuroimmunol. October 1;119(2):214-22.

Schwab J M, Seid K, Schluesener H J, 2001, Traumatic brain injury induces prolonged accumulation of cyclooxygenase-I expressing microglia/brain macrophages in rats, J Neurotrauma. 2001 September;18(9):881-90.

Sheehan D J, Hitchcock C A and Sibley C M, 1999, Current and emerging azole antifungal agents. Clin. Microbiol. Rev. 12,40-79.

Shen Y, Li R, McGeer E G, McGeer P L, 1997, Neuronal expression of mRNAs for complement proteins of the classical pathway in Alzheimer brain, Brain Research 769(2), 391-395.

Smedegard G, Bjork J, 1995, Sulfasalazine: mechanism of action in rheumatoid arthritis, Br J Rheumatol November;34 Suppl 2:7-15.

Stepkowski S M, Erwin-Cohen R A, Behbod F, Wang M E, Qu X, Tejpal N, Nagy Z S, Kahan B D, Kirken R A, 2002, Selective inhibitor of Janus tyrosine kinase 3, PNU156804, prolongs allograft survival and acts synergistically with cyclosporine but additively with rapamycin. Blood, 99, 680-689.

Strohmeyer R, Rogers J, 2001, Molecular and cellular mediators of Alzheimer's disease inflammation, Journal of Alzheimer's Disease 3, 131-157.

Tam R. C., Pai, B., Bard, J., Lim, C., Averett, D. R., Phan, U. T., Milovanoviv, T., 1999: Ribavirin polarizes human T cell responses towards a Type 1 cytokine profile. J. Hepatol. 30: 376-382

Tam, R. C., Ramasamy, K., bard, J., Pai, B., Lim, C., Averett, D. R., 2000: The Ribavirin Analog ICN 17261 Demonstrates Reduced Toxicity and Antiviral Effects with Retention of both Immunomodulatory Activity and Reduction of Hepatitis-Induced Serum Albumin Alanine Aminotransferase Levels. Antimicrob. Agents and Chemother. 44:1276-1283

Taupenot L, Ciesielski-Traska J, et al., 1996, Chromogranin A triggers a phenotypic transformation and the generation of nitric oxide in brain microglial cells, Neuroscience 72(2), 377-389.

Terai K, McGeer E G, McGeer P L, 1997, Neurons express proteins of the classical complement pathway in Alzheimer disease, Brain Research 769(2), 385-390.

Terrell C L and Hughes C E, 1992, Antifungal agents used for deep-seated mycotic infections. Mayo Clin Proc. 67,69-91.

Vere Hodge R. A., Sutton, D., Boyd, M. R., Hamden, M. R., Jarvest, R. L., 1989: Selection of an oral protherapeutic agent (BRL 42810; famciclovir) for the antiherpesvirus agent BRL 39123[9-(4-hydroxy-3-hydroxymethylbut-1-yl)guanine; penciclovir]. Antimicrob Agents Chemother 33(10):1765-73

Wahl B. C, Liptay S, Adler G, Schmid R M, 1998, Sulfasalazine: a potent and specific inhibitor of nuclear factor kappa, J Clin Invest March 1;101(5):1163-74.

Wallace J L, 1999, Distribution and expression of cyclooxygenase (COX) isoenzymes, their physiological roles, and the categorization of nonsteroidal anti-inflammatory therapeutic agents (NSAIDs), Am J Med December 13;107(6A):11S-16S; discussion 16S-17S.

Weinblatt M E, Dixon J A, Falchuk K R, 2001, Serious liver disease in a patient receiving methotrexate and leflunomide, Prescrire Int October;10(55):149; Arthritis Rheum November;43(11):2609-11.

Wilson C L, Ouellette A J, Satchell D P, Ayabe T, Lopez-Boado Y S, Stratman J L, Hultgren S J, Matrisian L M, Parks W C,1999, Regulation of intestinal alpha-defensin activation by the metalloproteinase matrilysin in innate host defense. Science October 1;286(5437):113-7.

Xu X, Shen J, Mall J W, Myers J A, Huang W, Blinder L, Saclarides T J, Williams J W, Chong A S, 1999, In vitro and in vivo antitumor activity of a novel immunomodulatory therapeutic agent, leflunomide: mechanisms of action, Biochem Pharmacol November 1;58(9):1405-13.

Patents:

| | | | |
|---|---|---|---|
| U.S. Pat. No. 3,926,955 | September 1972 | Burton et al. | 260/239 |
| U.S. Pat. No. 5,521,184 | April 1994 | Zimmermann | 514/252 |
| U.S. Pat. No. 5,721,277 | April 1995 | Tang | 514/646 |
| U.S. Pat. No. 6,271,383 | July 1999 | Gravestock | 546/209 |
| U.S. Pat. No. 6,136,796 | October 2000 | Kozak | 514/75 |
| U.S. Pat. No. 2001/0053782A1 | December 2000 | Blumenkopf et al. | 514/258 |
| U.S. Pat. No. 6,316,425 | November 2001 | Myhren et al. | 514/49 |
| WO9820876 | November 1996 | McCullough and Koch | C07D 521/00 |
| WO9821213 | November 1996 | McCullough et al. | A61K 31/495 |
| WO98/29437 | December 1997 | Young | 14/00 |
| WO9832762 | January 1997 | Myhren et al. | C07H 19/073 |
| WO9945008 | March 1998 | Hayase et al. | C07D 417/06 |
| WO0034281 | December 1998 | Whittaker et al | C07D 475/08 |
| WO0142246A2 | October 1999 | Blumenkopf et al. | C07D 487/04 |
| WO09/963937 | July 1999 | Griffin | |
| WO0114397A1 | August 1999 | Or et al. | C07H 17/08 |
| WO01/20331 | September 2000 | Dower et al | 33/566 |
| WO01/13957 | August 2000 | Rothbard | 47/48 |
| EP0013376B1 | May 1982 | Kammerer and Schleyerbach | C07D 261/18 |
| EP009944 | | Firestone | 31/13 |
| EP0627406A1 | October 1992 | Fujita et al. | C07C 215/10 |
| EP0632038B1 | January 1992 | Matsuoka et al. | C07C 475/08 |
| EP0676399A1 | December 1992 | Ohi and Suzuki | C07C 475/08 |
| JP05163293 | 514/252 | | |

What is claimed is:

1. A compound of the following formula:
T(L-C)ₘ,
wherein
T is a transportophore,
L is a bond or a linker having a molecular weight up to 240 dalton,
C is a non-antibiotic therapeutic agent, and
m is 1,2,3,4,5,6,7, or 8,
in which the transportophore has an immune selectivity ratio of at least 2, the transportophore is covalently bonded to the non-antibiotic therapeutic agent via the bond or the linker, and the compound has an immune selectivity ratio of at least 2.

2. The compound of claim 1, wherein the transportophore is an amphiphilic molecule having a pKa value of 6.5 to 9.5.

3. The compound of claim 1, wherein the transportophore is a cyclic or heterocyclic molecule.

4. The compound of claim 3, wherein the cyclic or heterocyclic molecule has an attached sugar.

5. The compound of claim 3, wherein the cyclic or herterocyclic molecule is a macrolactone or macroether.

6. The compound of claim 5, wherein the macrolactone or macroether has an attached sugar.

7. The compound of claim 3, wherein the cyclic or herterocyclic molecule is a macrolide or ketolide having an amino sugar.

8. The compound of claim 7, wherein the cyclic or herterocyclic molecule is a macrolide having mono-, di-, or tri-basic groups.

9. The compound of claim 1, wherein the compound is 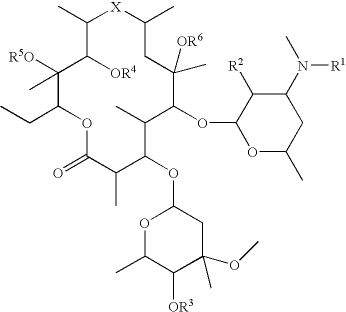
wherein
X=N(R⁷)—CH₂
CH₂—N(R⁷)
C(═O)
C(═NOR⁸)
CH(OR⁹)
CH(NR¹⁰R¹¹)
C(═NR¹²)
OC(═O)
C(═O)O
Y=independently linker
Z C(═O)—
CH(R¹⁶)
R¹=H
CH₃
(C₂-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
Y—R¹³
C(═O)—Y—R¹⁵
C(═O)—R¹⁵
R²=H
(1′,2′-cis)-OH
(1′,2′-trans)-OH
(1′,2′-cis)-OR¹⁵
(1′,2′-trans)-OR¹⁵
(1′,2′-cis)-SH
(1′,2′-cis)-S—Y—R¹³
or the R¹ and R² bearing atoms are connected via a —OC(═O)CHR¹⁶— element
R³=H
C(═O)—Y—R¹⁵
C(═O)—R¹⁵
R⁴=H
C(═O)—Y—R¹⁵
C(═O)—R¹⁵
R⁵=H
or R⁴, R⁵ are connected by Z
R⁶=H
CH₃
R⁷=H
CH₃
Y—R¹³
C(═O)—Y—R¹⁵
C(═O)—R¹⁵
R⁸=H
Y—R¹³
R¹³
C(═O)—R¹⁷
(C₁-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
wherein alkyl, alkenyl, alkynyl, aryl, and heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, —NR¹⁸R¹⁹, R¹⁸C(═O)—, R¹⁸C(═O)O—, R¹⁸OC(═O)O—, R¹⁸NHC(═O)—, R¹⁸C(═O)NH—, R¹⁸R¹⁹NC(═O)—and R¹⁸OC(═O)—
R⁹=H
(C₁-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
wherein alkyl, alkenyl, alkynyl, aryl, and heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, —NR¹⁸R¹⁹, R¹⁸C(═O)—, R¹⁸C(═O)O—, R¹⁸OC(═O)O—, R¹⁸NHC(═O)—, R¹⁸C(═O)NH—, R¹⁸R¹⁹NC(═O)—and R¹⁸OC(═O)—
R¹⁰, R¹¹=independently H
(C₁-C₁₀)alkyl
(C₁-C₁₀)alkenyl
(C₁-C₁₀)alkynyl
(C₁-C₈)[(C₁-C₄)alkoxy]alkyl
(C₁-C₉)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
or R¹⁰=H and R¹¹=—Y—R¹³
C(═O)—Y—R¹⁵, —C(═O)—R¹⁵
R¹²=H
(C₁-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
Y—R¹³
R¹³=independently, therapeutic agent
R¹⁵=independently, therapeutic agent
R¹⁶=H
CH₃
(C₂-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
Y—R¹³,
R¹⁷=O—R²⁰-aryl
optionally substituted by —X′-Y— therapeutic agent, X′-therapeutic agent wherein X′ is S, O, or NH
R¹⁸, R¹⁹=independently H
(C₁-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
R²⁰=independently,
Halogen
(C₁-C₃)alkyl
NO₂
CN
OCH₃
N(CH₃)₂
N₃
SH
S(C₁-C₄)alkyl.

10. The compound of claim 1, wherein the compound is 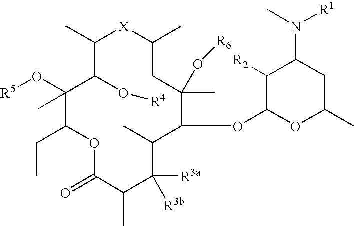
wherein:
X=N(R⁷)—CH₂
CH₂—N(R⁷)
C(═O)
C(═NOR⁸)
CH(OR⁹)
CH(NR¹⁰R¹¹)
C(═NR¹²)
OC(═O)
C(═O)O
Y=independently, linker
Z=C(═O)—
CH(R⁶)—
R¹=H
CH₃
(C₂-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
Y—R¹³
C(═O)—Y—R¹⁵
C(═O)—R¹⁵
S(═O)ₖ(C₁-C₁₀)alkyl
S(═O)ₖ(C₁-C₁₀)alkenyl
S(═O)ₖ(C₁-C ₁₀)alkynyl
S(═O)ₖ(C₆-C₁₀)aryl
S(═O)ₖ(C₂-C₉)heteroaryl
S(═O)ₖ—Y—R¹⁵
S(═O)ₖ—R¹⁵
wherein k is 0, 1 or 2 and alkyl, alkenyl, alkynyl, cycloalkyl, heterocycloalkyl, aryl and heteroaryl can optionally be substituted by one to three halogen, cyano, hydroxy, (C₁-C₄)alkyloxy, nitro, (C₁-C₆)alkyl, (C₁-C₆)alkenyl, (C₁-C₆)alkynyl, (C₃-C₇)cycloalkyl, (C₁-C₆)heterocycloalkyl, (C₆-C₁₀)aryl, (C₁-C₉)heteroaryl, NR¹⁸R¹⁹, R¹⁸C(═O)—, R¹⁸C(═O)O—, R¹⁸OC(═O)—, R¹⁸C(═O)NH—, R¹⁸NHC(═O)—, R¹⁸R¹⁹NC(═O)— or R¹⁸OC(═O)—O—
R²=H
(1′,2′-cis)-OH
(1′,2′-trans)-OH
(1′,2′-cis)-OR¹⁵
(1′,2′-trans)-OR¹⁵
(1′,2′-cis)-SH
(1′,2′-cis)-S—Y—R¹³
or the R¹ and R² bearing atoms are connected via a —OC(═O)CHR¹⁶— element
R^{3a}, R^{3b}=independently H
R¹
OH
OR¹¹
NR¹⁰R¹¹
or R^{3a}=R^{3b}=(═O),
(═NR¹)
O(CH₂)ₖO— wherein k is 2 or 3
R⁴=H
C(═O)—Y—R¹⁵
C(═O)—R¹⁵
R⁵=H
or R⁴, R⁵ are connected by —Z—
R⁶=H
CH₃
R⁷=H
CH₃
Y—R¹³
C(═O)—Y—R¹⁵
C(═O)—R¹⁵
R⁸=H
Y—R¹³
C(═O)—R¹⁷
R⁹=H
(C₁-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
R¹⁰, R¹¹=independently H
(C₁-C₁₀)alkyl
(C₁-C₁₀)alkenyl
(C₁-C₁₀)alkynyl
(C₃-C₁₀)cycloalkyl
(C₁-C₉)heterocycloalkyl
(C₆-C₁₀)aryl
(C₂-C₉)heteroaryl
wherein alkyl, alkenyl, alkynyl, cycloalkyl, heterocycloalkyl, aryl, heteroaryl are optionally substituted by one to three halogen, cyano, hydroxy, (C₁-C₄)alkyloxy, nitro, (C₁-C₆)alkyl, (C₁-C₆)alkenyl, (C₁-C₆)alkynyl, (C₃-C₇)cycloalkyl, (C₁-C₆)heterocycloalkyl, (C₆-C₁₀)aryl, (C₁-C₉)heteroaryl, NR¹⁸R¹⁹, R¹⁸C(═O)—, R¹⁸C(═O)O—, R¹⁸OC(═O)—, R¹⁸C(═O)NH—, R¹⁸NHC(═O)—, R¹⁸R¹⁹NC(═O)— or R¹⁸OC(═O)—O—
or R¹⁰=H and
R¹¹=Y—R¹³
C(═O)—Y—R¹⁵
C(═O)—R¹⁵
S(═O)ₖ(C₁-C₁₀)alkyl
S(═O)ₖ(C₁-C ₁₀)alkenyl
S(═O)ₖ(C₁-C₁₀)alkynyl
S(═O)ₖ(C₆-C₁₀)aryl
S(═O)ₖ(C₂-C₉)heteroaryl
S(═O)ₖ—Y—R¹⁵
S(═O)ₖ—R¹⁵
wherein k is 0, 1 or 2 and alkyl, alkenyl, alkynyl, cycloalkyl, heterocycloalkyl, aryl and heteroaryl can be substituted as defined above.
R¹²=H
(C₁-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
Y—R¹³
R¹³=independently, therapeutic agent
R¹⁵=independently, therapeutic agent
R¹⁶=H
CH₃
(C₂-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
(C₁-C₄)alkyliden-NR¹⁸R¹⁹
Y—R¹³
R¹⁷=O—R²⁰-aryl
optionally substituted by —X′—Y'a therapeutic agent, X′-a therapeutic agent wherein X′ is
S, O, NH
R¹⁸, R¹⁹=independently H
(C₁-C₁₀)alkyl
(C₁-C₁₀)alkenyl
(C₁-C₁₀)alkynyl
(C₁-C₈)[(C₁-C₄)alkoxy]alkyl
(C₁-C₈)[(C₁-C₄)alkoxy]alkenyl
(C₆-C₁₀)aryl-(C₁-C₅)alkyl
(C₂-C₉)heteroaryl-(C₁-C₅)alkyl
R²⁰=independently,
Halogen
(C₁-C₃)alkyl
NO₂
CN
OCH₃
N(CH₃)₂
N₃
SH
S(C₁-C₄)alkyl.

11. The compound of claim 1, wherein the compound is 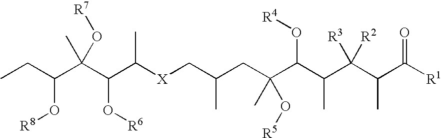
wherein
X=N(R⁹)—CH₂
CH₂—N(R⁹)
C(═O)
C(═NOR¹⁰)
C(OR¹¹)H
CH(NR¹²R¹³)
C(═NR¹⁴)
OC(═O)
C(═O)O
Y=independently, linker
R¹=OR¹⁷
NR¹⁷ R¹⁸,
or R¹ is connected to the oxygen bearing R⁴ or R⁵ forming a lactone or is connected to a suitable substituent in R² forming a lactone or lactam,
R²=O-2-cladinosyl 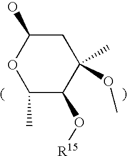
H
X′, wherein X′=halogen
azido
nitro
cyano
OR¹⁷
OR²²
NR¹⁷ R¹⁸
SR¹⁷ (C₁-C₆)alkyl
(C₁-C₆)alkenyl
(C₁-C₆)alkynyl
(C₃-C₁₀)cycloalkyl
(C₁-C₉)heterocycloalkyl
(C₆-C₁₀)aryl
(C₁-C₉)heteroaryl
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, RTeC(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y— therapeutic agent or -therapeutic agent,
R³=H
(C₁-C₆)alkyl
(C₁-C₆)alkenyl
(C₁-C₆)alkynyl
(C₃-C₁₀)cycloalkyl
(C₁-C₉)heterocycloalkyl
(C₆-C₁₀)aryl
(C₁-C₉)heteroaryl
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, or R²⁰R²¹N—
R⁴=O-2-desosaminyl 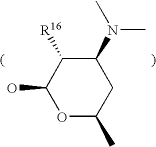
H
C(═O)R¹⁷
Y— therapeutic agent
therapeutic agent
S(═O)₂R¹⁷ providing R¹⁷ is not hydrogen
C(═O)NR¹⁷R¹⁸(C₁-C₆)alkyl
(C₁-C₆)alkenyl
(C₁-C₆)alkynyl
(C₃-C₁₀)cycloalkyl
(C₁-C₉)heterocycloalkyl
(C₆-C₁₀)aryl
(C₁-C₉)heteroaryl
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y— therapeutic agent or -therapeutic agent,
or R⁴ is connected to a suitable R² containing a N or a O by —C(═O), S(═O),
wherein n=1 or 2, —CR²⁰R¹⁷—, CR²⁰(—Y— therapeutic agent)-, —CR²⁰(- therapeutic agent)-forming in dependence of R² a 6 or 7-membered ring,
R⁵=R²⁰
C(═O)R²⁰
or R⁴, R⁵ are connected by C(═O), S(═O)ₙ wherein n=1 or 2, —CR²⁰R¹⁷—, CR²⁰(—Y—therapeutic agent)-, —CR²⁰(-therapeutic agent)-
R⁶, R⁸=independently H
(C₁-C₆)alkyl
(C₁-C₆)alkenyl
(C₁-C₆)alkynyl
(C₃-C₁₀)cycloalkyl
(C₁-C₉)heterocycloalkyl
(C₆-C₁₀)aryl
(C₁-C₉)heteroaryl
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(O)—, R²⁰C(═O)NH—, R²⁰R²¹NC(═O)—, and R²⁰OC(═O)O—, —Y— therapeutic agent or -therapeutic agent,
or R⁶, R⁸=independently —C(═O)R¹⁷, —Y— therapeutic agent, -therapeutic agent, —S(═O)₂R¹⁷ providing R¹⁷ is not hydrogen, —C(═O)NR¹⁷R¹⁸,
R⁷=H
(C₁-C₆)alkyl
(C₁-C₆)alkenyl
(C₁-C₆)alkynyl
(C₃-C₁₀)cycloalkyl
(C₁-C₉)heterocycloalkyl
(C₆-C₁₀)aryl
(C₁-C₉)heteroaryl
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y— therapeutic agent or -therapeutic agent,
or two of each R⁶, R⁷, R⁸ are connected by —C(═O), S(═O)ₙ wherein n=1 or 2, —CR²⁰R¹⁷—, CR²⁰(—Y— therapeutic agent)-, —CR²⁰(-therapeutic agent)-,
R⁹=H
CH₃
Y-therapeutic agent
therapeutic agent
(C₁-C₆)alkyl
(C₁-C₆)alkenyl
(C₁-C₆)alkynyl,
wherein alkyl, alkenyl, alkynyl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y—therapeutic agent or -therapeutic agent,
R¹⁰=C(═O)-aryl
therapeutic agent,
H
(C₁-C₆)alkyl
(C₁-C₆)alkenyl
(C₁-C₆)alkynyl,
wherein alkyl, alkenyl, alkynyl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y— therapeutic agent or -therapeutic agent
R¹¹=H
(C₁-C₆)alkyl
(C₁-C₆)alkenyl
(C₁-C₆)alkynyl,
wherein alkyl, alkenyl, alkynyl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, R²⁰OC(═O)O—, —Y— therapeutic agent or -therapeutic agent,
or R¹²=—Y— therapeutic agent, -therapeutic agent, —C(═O)R¹⁷
R¹², R¹³=independently H
(C₁-C₆)alkyl
(C₁-C₆)alkenyl
(C₁-C₆)alkynyl
(C₃-C₁₀)cycloalkyl
(C₁-C₉)heterocycloalkyl
(c₆-C₁₀)aryl
(C₁-C₉)heteroaryl,
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, R²⁰OC(═O)O—, —Y— therapeutic agent or -therapeutic agent,
or R¹², R¹³=independently —C(═O)R¹⁷, —Y— therapeutic agent, -therapeutic agent, —S(═O)₂R¹⁷ providing R¹⁷ is not hydrogen, —C(═O)NR¹⁷R¹⁸
R¹⁴=therapeutic agent
H
(C₁-C₆)alkyl
(C₁-C₆)alkenyl
(C₁-C₆)alkynyl
(C₃-C₁₀)cycloalkyl
(C₁-C₉)heterocycloalkyl
(C₆-C₁₀)aryl
(C₁-C₉)heteroaryl
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²¹NHC(═O)—, R²⁰C(═O)NH—, R²¹R²¹NC(═O)—, R²⁰OC(═O)O—, —Y— therapeutic agent or -therapeutic agent,
R¹⁵=H
C(═O)R¹⁷
Y— therapeutic agent,
therapeutic agent,
S(═O)₂R¹⁷ providing R¹⁷ is not hydrogen
C(═O)NR⁷ R¹⁸
(C₁-C₆)alkyl
(C₁-C₆)alkenyl
(C₁-C₆)alkynyl
(C₃-C₁₀)cycloalkyl
(C₁-C₉)heterocycloalkyl
(C₆-C₁₀)aryl
(C₁-C₉)heteroaryl,
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y— therapeutic agent or -therapeutic agent,
R¹⁶=H
OR¹⁷
OR²²
R¹⁷, R¹⁸=independently H
(C₁-C₆)alkyl
(C₁-C₆)alkenyl
(C₁-C₆)alkynyl
(C₃-C₁₀)cycloalkyl
(C, —C)heterocycloalkyl
(C₆-C₁₀)aryl
(C₁-C₉)heteroaryl
wherein alkyl, alkenyl, alkynyl, cycloalkyl, heterocycloalkyl, aryl, heteroaryl groups are optionally substituted by one to five substituents selected independently from halogen, (C₁-C₄)alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)—, R²⁰C(═O)O—, R²⁰OC(═O)—, R²⁰NHC(═O)—, R²⁰C(═O)NH—, R²⁰R²¹NC(═O)—, and R²⁰OC(═O)O—, —Y— therapeutic agent or -therapeutic agent,
or provided that connected to a nitrogen, R¹⁷, R¹⁸ may form a cyclic structure of 4 to 7 members (including the nitrogen). R¹⁷ and R¹⁸ then can represent a fragment from the type of -[C(AB)]ₘ-Ξₙ-[C(DE)]ₒ-Ψₚ-[C(GJ)]_{q} wherein m, n, o, p and q independently are 0, 1, 2, 3, 4, 5, or 6, Ξ and Ψ independently are —O—, —S—, —NK— and A, B, D, E, G, J, and K independently are hydrogen, (C₁-C₄) alkyl, (C₁-C₄)alkenyl, (C₁-C₄)alkynyl, (C₃-C₇)cycloalkyl, (C₁-C₆)heterocycloalkyl, (C₆-C₁₀)aryl, (C₁-C₉)heteroaryl, (C₁-C₄)alkoxy, hydroxy, nitro, cyano, azido, mercapto, R²⁰R²¹N—, R²⁰C(═O)O—, R²⁰C(═O)O—, R²⁰OC(═O)—, R²⁰NHC(═O)—, R²⁰C(═O)NH—, R²⁰R²¹NC(═O)—, and R²⁰OC(═O)O—
R²⁰, R²¹=independently H
(C₁-C₆)alkyl
R²²=C(═O)R¹⁷
Y— therapeutic agent
therapeutic agent,
S(═O)₂R¹⁷ providing R¹⁷ is not hydrogen, —C(═O)NR¹⁷R¹⁸.

12. The compound of claim 1, wherein the compound is 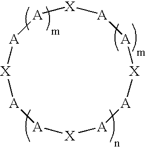
wherein:
m=independently, 0, 1, 2, 3
n=0-7
X=independently, O
S
Se
NR¹
PR¹
with the proviso, that at least one X=—NR¹—
A=independently, CH₂
CHR²
CR²R³
C(═O)
with the proviso, that at least one X=NR¹— is not an amide
R¹=independently, H
(C₁-C₁₀)alkyl, optionally substituted by fluoro, cyano, R⁴, R⁴O₂C, R⁴C(═O)NH and R⁴S(═O)ₖ wherein k is 0, 1 or 2
R⁴C(═O), R⁴S(═O)ₖ wherein k is 0, 1 or 2
R², R³=independently NH₂
NHR¹
NR¹R⁵
OH,
OR⁴
R⁴C(═O) (C₁-C₆)alkyl
(C₂-C₁₂)alkenyl
(C₂-C₁₂)alkynyl
(C₃-C₁₀)cycloalkyl(C₁-C₆)alkyl
(C₂-C₉)heterocycloalkyl(C i —C₆)alkyl
(C₆-C₁₀)aryl(C₁-C₆)alkyl
(C₂-C₉)heteroaryl(C₁-C₆)alkyl,
wherein the alkyl, alkenyl, alkynyl, cycloalkyl, heterocycloalkyl, aryl, and heteroaryl groups are optionally substituted by one to three halo, (C₁-C₄)alkoxy, hydroxy, nitro, cyano, —C(═O)—OR⁸, —C(═O)N(H)R⁸, (C₆-C₁₀)aryl, (C₂-C₉)heteroaryl, N*R⁵R⁶R⁷ wherein * is no or a positive charge, one or two of R², R³ can be a directly coupled therapeutic agent,
R⁴=independently,
NH2
NHR⁹
NR⁹R⁵
OH
OR⁹
(C₁-C₆)alkyl
(C₂-C₁₂)alkenyl
(C₂-C₁₂)alkynyl
(C₃-C₁₀)cycloalkyl(C₁-C₆)alkyl
(C₂-C₉)heterocycloalkyl(C₁-C₆)alkyl
(C₆-C₁₀)aryl(C₁-C₆)alkyl
(C₂-C₉)heteroaryl(C₁-C₆)alkyl,
wherein the alkyl, alkenyl, alkynyl, cycloalkyl, heterocycloalkyl, aryl, and heteroaryl groups are optionally substituted by one to three halo, (C₁-C₄)alkoxy, hydroxy, nitro, cyano, R⁸, —C(═O)—OR⁸, —C(═O)N(H)R⁸, (C₆-C₁₀)aryl, (C₂-C₉)heteroaryl, N*R⁵R⁶R⁷ wherein * is no or a positive charge, or
a therapeutic agent,
R⁵, R⁶=independently H
(C₁-C₆), optionally substituted by hydroxy
(C₆-C₁₀)aryl
(C₂-C₉)heteroaryl
R⁷=independently,
lone electron pair
CH₃
C₂H₅
C₃H₇
CH₂—C₆H₅
R⁸=independently, therapeutic agent
R⁹=independently,
(C₁-C₆)alkyl
(C₂-C₁₂)alkenyl
(C₂-C₁₂)alkynyl
(C₃-C₁₀)cycloalkyl(C₁-C₆)alkyl
(C₂-C₉)heterocycloalkyl(C₁C₆)alkyl
(C₆-C₁₀)aryl(C₁-C₆)alkyl or
(C₂-C₉)heteroaryl(C₁-C₆)alkyl,
wherein the alkyl, alkenyl, alkynyl, cycloalkyl, heterocycloalkyl, aryl, and heteroaryl groups are optionally substituted by one to three halo, (C₁-C₄)alkoxy, hydroxy, nitro, cyano, R⁸, —C(═O)—OR⁸, —C(═O)N(H)R⁸, (C₆-C₁₀)aryl, (C₂-C₉)heteroaryl, N*R⁵R⁶R⁷ wherein * is no or a positive charge, or
a therapeutic agent.

13. The compound of claim 1, wherein the linker is
(C₁-C₅)alkyl,
(C₁-C₅)alkenyl,
(C₁-C₈)alkynyl,
(C₃-C₁₀)cycloalkyl,
(C₆-C₁₀)aryl,
(C₂-C₉)heteroalkyl, or
(C₂-C₉)heteroaryl,
wherein alkyl-, alkenyl, alkynyl, cycloalkyl, aryl or heteroaryl spacing elements are optionally substituted by (C₁-C₆)alkyl, 1-4 halogens, (C₁-C₄)alkoxy, (C₁-C₄)alkoxycarbonyl, hydroxy, amino, (C₁-C₄)alkylamino, (C₁-C₄)dialkylamino, (C₃-C₁₀)cycloalkyl, (C₁-C₆)alkylcarbonyloxy, (C₁-C₆)alkylcarbonylamido, (C₁-C₄)alkylamidocarbonyl, (C₁-C₄)dialkylamidocarbonyl, nitro, cyano, (C₁-C₄)alkylimino, mercapto or (C₁-C₄)alkylmercapto.

14. The compound of claim 1, wherein the non-antibiotic therapeutic agent is an anti-inflammatory agent.

15. The compound of claim 1, wherein the anti-inflammatory agent is a protein kinase inhibitor, a protease inhibitor, or an HMGCoA reductase inhibitor.

16. The compound of claim 1, wherein the non-antibiotic therapeutic agent is an anti-infectious agent.

17. The compound of claim 1, wherein the anti-infectious agent is a protease inhibitor.

18. The compound of claim 1, wherein the non-antibiotic therapeutic agent is an anti-cancer agent.

19. The compound of claim 1, wherein the non-antibiotic therapeutic agent is a fluorescent molecule useful in diagnostic or exploratory applications.

20. The compound of claim 1, wherein the non-antibiotic therapeutic agent is an immune-suppressant agent.

21. The compound of claim 1, wherein the immune-suppressant agent is an analog of vitamin D or a statin.

22. The compound of claim 1, wherein the non-antibiotic therapeutic agent is an agent for treating a hematopoietic disorder.

23. The compound of claim 1, wherein the non-antibiotic therapeutic agent is an agent for treating a metabolic disease.

24. The compound of claim 1, wherein the metabolic disease is excessive coagulation, or hypercholesterolemia.

25. A pharmaceutical composition comprising a compound of claim 1 and a pharmaceutically acceptable carrier.

26. A method of treating an inflammatory disorder, comprising administering to a subject in need thereof an effective amount of a compound of claim 1, wherein the non-antibiotic therapeutic agent is an anti-inflammatory agent.

27. A method of treating an infectious disease, comprising administering to a subject in need thereof an effective amount of a compound of claim 1, wherein the non-antibiotic therapeutic agent is an anti-infectious agent.

28. A method of treating cancer, comprising administering to a subject in need thereof an effective amount of a compound of claim 1, wherein the non-antibiotic therapeutic agent is an anti-cancer agent.

29. A method of treating allergy, comprising administering to a subject in need thereof an effective amount of a compound of claim 1, wherein the non-antibiotic therapeutic agent is an allergy-suppressive agent.

30. A method of treating an immune disorder, comprising administering to a subject in need thereof an effective amount of a compound of claim 1, wherein the non-antibiotic therapeutic agent is an immune-suppressant agent.
